(19) United States
(12) Patent Application Publication
(10) Pub. No.: US 2025/0178943 A1
(43) Pub. Date: Jun. 5, 2025

(54) SYSTEM, METHOD AND DEVICE FOR TREATING A LIQUID

(71) Applicant: Ellen Medical Devices Pty Ltd, Sydney (AU)

(72) Inventor: Vincent Joseph Garvey, Colby (IM)

(21) Appl. No.: 18/845,837

(22) PCT Filed: Mar. 11, 2022
(86) PCT No.: PCT/AU2022/050210

§ 371 Date: Sep. 10, 2024

(30) Foreign Application Priority Data
Mar. 11, 2021 (AU) 2021900704

(51) Int. Cl. C02F 9/20; C02F 101/32; C02F 103/02

(52) U.S. Cl. CPC C02F 9/20; C02F 2101/322; C02F 2103/026; C02F 2201/007; C02F 2209/02; C02F 2209/03; C02F 2301/066; C02F 2301/08

(57) ABSTRACT

A multistage treatment system for a liquid, the system including a first stage with a first boiling vessel and a first heater configured to boil the liquid in the first boiling vessel at a first pressure. The boiling of the liquid generates vapour including volatile contaminants. A first vapour outlet is configured to vent the vapour from the first boiling vessel. A liquid outlet provides for fluid flow from the first boiling vessel to a subsequent stage. A second stage comprises a second boiling vessel configured to receive liquid from the liquid outlet and a second heater to boil liquid in the second boiling vessel to generate vapour. A condenser receives the vapour from the second boiling vessel and condenses the vapour to a liquid that is treated. An outlet is configured to dispense treated liquid.

### FIELD OF THE INVENTION

The invention relates to processes for treatment of liquids. In particular, the invention relates to multistage treatment of liquids to remove contaminants. Specific forms of the invention relate to a device to treat a fluid such as water in two or more stages to remove volatile contaminants and particulates.

### BACKGROUND OF THE INVENTION

The distillation of liquids is a known and effective purification process. In particular, the distillation of water is a known technique for removing contaminants.

Conventional single-stage water distillation can remove most impurities from water. Typically, the compounds removed include sodium, calcium, magnesium, and other dissolved solids such as iron and manganese fluoride and nitrate. Distillation also removes other organic compounds such as heavy metals (e.g. lead), chlorine, chloramines and radionuclides. The boiling process will disable bacteria and other organic forms, but the remaining endotoxins can be removed by distillation. Unfortunately, there are a number of drawbacks associated with conventional single-stage distillation:
1. Some organic compounds are not easily removed by single-stage distillation due to their chemical properties, and in particular, their boiling points. Certain pesticides, volatile solvents and volatile organic compounds (VOCs), such as benzene and toluene, will vaporize along with the water as it is boiled in the distiller because their boiling points are near or below that of water. Similarly, such compounds condense together with the water vapour and remain as a contaminant. This necessitates an additional process for their removal prior to condensation.
2. Boiling water during distillation inactivates microorganisms such as bacteria, viruses and protozoan cysts. After the distillation process, the newly distilled water may be re-contaminated with bacteria or other micro-organisms via the water outlet.
3. Contaminants and impurities collect within the distiller, most commonly in the evaporation section. This build-up of contaminants will eventually result in device failure without a regular maintenance regime.
4. It is known to introduce a vent between the evaporator and the condenser for removing VOCs prior to condensation. In reality, only a small proportion of VOCs are removed via the vent. In fact, the amount of VOCs removed via the vent is a small proportion of the amount of steam also vented. Distillation systems for use in medical devices or similar, require the VOCs to be (effectively) eliminated from the distilled water.

To address these issues, a process of double-distillation may be used. Unfortunately, this is often ineffective as the VOCs that evaporate in the first stage distillation will recondense, and then simply recondense a second time after the second stage distillation.

In some industrial scale systems, a column of pressurised steam is used as preheated process water. The water is further sprayed through the steam increasing its surface contact area. This dual process is effective in degassing the incoming water. Steam has a very high affinity for dissolution in water which in turn drives out the dissolved VOCs. The VOCs are collected and removed so that only distilled water remains. By way of example, WO 2018/148247 entitled “Water Treatment and Desalinisation” discloses an industrial scale multistage treatment system that involves a complex system of pumps and valves and relies on a steam generator to provide steam to the system. These types of systems also require industrial scale power supplies and are not able to be powered by conventional single phase mains power supplies available in a domestic environment.

Unfortunately, this technique does not scale down to a small or domestic distillation-scale system. The majority of the domestic appliances need to operate on mains electrical power (i.e. 1,000 W to 3,400 W) and cannot be configured to degas the water in an economically practical way.

The Applicant has developed a domestic scale fluid treatment device as described in WO 2017/109760 A1 entitled “Treatment Fluid Preparation System”. This device uses a small compressor to pressurise vapour, thus facilitating the degas process using only the power provided by a domestic electrical outlet. However, while providing many advantages, the incorporation of a vapour compressor into the water treatment system adds complexity and production costs.

Any reference herein to a patent document or other matter which is given as prior art is not to be taken as an admission that that document or matter was known or that the information it contains was part of the common general knowledge as at the priority date of any of the claims.

Throughout the description and claims of the specification, any one of the terms “comprising”, “comprised of” or “which comprises” and their variations are open terms that mean including at least the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting only of elements A and B. Any one of the terms including or which includes or that includes as used herein is also an open term that also means including at least the elements/features that follow the term, but not excluding others. Thus, including is synonymous with and means comprising.

### SUMMARY OF THE INVENTION

According to a first aspect, the present invention provides a multistage treatment system for a liquid, the system comprising:
a first stage comprising a first boiling vessel and a first heater configured to boil the liquid in the first boiling vessel at a first pressure, the first pressure being greater than ambient pressure, such that the boiling liquid generates vapour including volatile contaminants, a first vapour outlet configured to vent the vapour from the first boiling vessel, and a liquid outlet for fluid flow from the first boiling vessel to a subsequent stage; and,
a second stage comprising a second boiling vessel having a liquid inlet to receive liquid from the first liquid outlet and a second heater to boil liquid in the second boiling vessel to generate vapour, the vapour being output via a second vapour outlet in the second boiling vessel;
a condenser to receive the vapour from the second vapour outlet and condense the vapour to a liquid that is treated, and
a primary outlet configured to dispense treated liquid.

In some embodiments, the system includes a heat exchanger having a first end connected to the first vapour outlet and a second end connected to ambient air. The heat exchanger preferably includes a first temperature sensor disposed at a location in the heat exchanger where the temperature is approximately half way between an internal temperature at the first boiling vessel and an output temperature.

The first stage performs a degassing process to remove volatile gas contaminants that get trapped in steam and exit through the first vapour outlet. The second stage performs a distilling operation to remove larger particulate contaminants. The design allows for a continuous flow process to be performed wherein treated liquid can be output from the system even while untreated water is being processed by the system. The system can be powered from a standard single phase mains power supply and be hand-portable in size. The in-line design of the system allows for additional processing stages such as filtering and degassing to be introduced before, between or after the two stages described above.

By ‘mains’ power, it is intended to mean a general-purpose alternating-current (AC) electric power supply that is supplied to domestic and commercial premises through the standard power grid and available through a standard wall plug. Mains power may also be referred to as utility power, domestic power or wall power. Example mains power systems use 230-240 v at 50 Hz or 120 V at 60 Hz. Devices operating off mains power typically consume power less than about 3,400 W.

By ‘portable’, it is intended to mean a device that is able to be moved by a single able bodied person. This may be similar in dimensions to a household appliance such as a fridge or microwave. By way of example, a portable fluid treatment device may weigh less than 40 kg and have dimensions less than 600 mm by 600 mm by 900 mm.

The first vapour outlet may include a first outlet valve configured open at or above the first pressure. The second boiling vessel may be configured to generate vapour at a second pressure. The second pressure may be greater than ambient pressure and less than the first pressure. The second boiling vessel may include a steam outlet having an outlet valve that is configured to open at a predefined threshold pressure to release steam.

Each stage of the treatment system preferably operates at a pressure greater than ambient pressure, with the pressure in the first stage preferably greater than subsequent stages. The high vapour pressure increases the boiling temperature and more effectively removes VOCs from the liquid.

A multistage treatment system according to the invention continuously removes volatile contaminants by venting to the ambient outside environment. The final stage condenses the vapour to provide a treated liquid at the outlet valve with the required purity. The liquid remaining in any of the stages may be subsequently removed to prevent the build-up of scale and contaminants. With each stage operating above ambient pressure, the valves and positive displacement pumps effectively seal the treatment system when the power is interrupted and internal pressures suddenly drop. The system can be in the form of a relatively inexpensive domestic appliance that is not technically complex to operate.

Preferably the first stage includes a first fluid supply device for drawing the liquid from a source and supplying liquid to the first boiling vessel. The first fluid supply device may be a pump, a solenoid valve or other device configured to supply fluid to the first stage in a controlled manner. The fluid source may be a reservoir, container or mains water source.

Preferably, the first boiling vessel includes a first liquid sensor for sensing when the first heater is immersed in the liquid. The first fluid supply device is preferably selectively deactivated when the first liquid sensor senses the first heater is not immersed in the liquid.

The first boiling vessel may include a second liquid sensor disposed at a higher position than the first liquid sensor in the first boiling vessel. Output from the second liquid sensor may be used to control the first fluid supply device.

Preferably, the second liquid sensor is configured to generate a signal that enables a continuously variable output signal to be generated by a controller that is indicative of a mean liquid level in the first boiling vessel.

In a further preferred form, the second liquid sensor includes a capacitor with a dielectric material between capacitor electrodes such that an amount of the dielectric material between the electrodes varies with changes in the liquid level, thereby changing the output signal.

In some embodiments, the first and/or second liquid sensor includes at least one electrically conductive sensing electrode capable of sensing a level of liquid based on its conductivity.

In some embodiments, the first and/or second liquid sensor includes a pair of electrically conductive sensing electrodes positioned to sense liquid levels of different height based on conductivity of the liquid.

Preferably, the first stage has a liquid temperature sensor and a vapour temperature sensor. During use, output from the liquid temperature sensor and the vapour temperature sensor is used for feedback control of the first heater. In some embodiments, control of the first heater is based at least in part on a temperature sensed by the first temperature sensor.

In an embodiment preferred for its simplicity, the first stage includes a degas module, and the second stage includes an evaporator module in fluid communication with the degas module via a second fluid supply device. The second fluid supply device may be a pump. The second fluid supply device preferably draws the liquid from the first boiling vessel to the second boiling vessel. The evaporator module preferably includes one or more evaporator liquid sensors and wherein one of the evaporator liquid sensors is positioned to detect when the second heater is immersed or covered in the liquid.

The second fluid supply device is preferably able to be controlled in reverse to flush liquid and contaminants from the second boiling vessel along an output path at specific intervals.

In some embodiments, the system includes a third fluid supply device in fluid communication with the second boiling vessel and configured to move liquid and contaminants from the second boiling vessel at specific intervals.

In some embodiments, the second boiling vessel includes an outlet tube connected to a pressure release valve that opens when a pressure in the second boiling vessel reaches a pressure threshold to expel liquid and contaminants from the second boiling vessel.

Preferably, the second fluid supply device is disabled until the first temperature sensor in the degas module reaches a predefined threshold temperature and a predefined amount of time has passed at or above this threshold temperature.

In a commercially relevant form of the invention, the liquid is water and the system generates a flow rate between 0.8 kilograms per hour to 1.3 kilograms per hour of water out of the condenser using less than 800 W during steady state continuous flow.

In some embodiments, the system is configured to operate on a total input power of less than or equal to 3,400 W. In other embodiments, the system is configured to operate on a total input power of less than or equal to 2,400 W. In further embodiments, the system is configured to operate on a total input power of less than or equal to 1,500 W.

Preferably, the first and second fluid supply devices are positive displacement pumps configured to prevent liquid flow when not operating.

Preferably, the positive displacement pumps are peristaltic pumps.

Preferably, the system further comprises an output heat exchanger for receiving outlet water flow from the condenser to heat inlet water flow to the degas module.

Preferably, the output heat exchanger has a conduit arranged in a serpentine flow path for the outlet water flow, and an external cover encasing the conduit defining a flow path over the conduit exterior for the inlet water flow.

Preferably, the flow path defined by the external cover is a serpentine flow path around the conduit and the inlet water flow direction is counter to the outlet water direction.

Preferably, the condenser outlet valve is configured for sealed fluid connection to a container containing concentrated solutes to form a treatment solution with the water treated by the system.

Preferably the system is powered by a mains power supply. The mains power supply may supply a voltage of 220 V to 240 V at 50 Hz or 110 V to 120 V at 60 Hz. More preferably, the mains power supply is a conventional single phase wall outlet that allows drawing of a maximum of 20 amps.

In some embodiments, the system includes an output temperature sensor positioned in an outlet fluid flow of the condenser. The output temperature sensor is configured to sense a temperature of fluid from the condenser. The second heater is responsive to a signal from the output temperature sensor to increase or decrease energy input to the liquid in the second boiling vessel.

In some embodiments, the condenser includes a fan and the condenser fan is responsive to a signal from the output temperature sensor to increase or decrease the fan speed.

In some embodiments, the system includes a third heater disposed between the second boiling vessel and the condenser configured to superheat steam entering the condenser.

Preferably the system is configured to provide a continuous flow process in which treated liquid is dispensed concurrently while liquid is being processed in the first and second stages.

In some embodiments, the system includes a mechanically actuatable sterile coupling device for moving the outlet into sterile coupling engagement with an inlet of a container of dried medicament.

The sterile coupling device may be electromechanically actuatable into sterile coupling engagement with an inlet of a container of dried medicament in response to a control signal.

In some embodiments, the system includes a weight sensor for sensing the weight of the container during a filling process.

In some embodiments, the system includes a first fluid level sensor for sensing a first level of fluid in the boiling vessel and generating a first sensor signal and a second fluid level sensor for sensing a second level of fluid in the boiling vessel and generating a second sensor signal, the second level being higher than the first level. The system may additionally include a controller responsive the first and second sensor signals to control the speed of the first pump and/or second pump.

In some embodiments, the controller is responsive to the first sensor signal to activate the first or second heater when fluid is detected at or above the first level in the corresponding first or second boiling vessel.

In some embodiments, the controller is responsive to the second sensor signal to deactivate the first or second pump when fluid is detected at or above the second level in the corresponding first or second boiling vessel.

In some embodiments, the controller is configured to monitor the second sensor signal over a period of time to detect splashing of the fluid at the second level when the fluid level is approaching the second level.

In some embodiments, the controller is configured to detect a ratio of ‘on’ time to total time of the second sensor signal, wherein ‘on’ time indicates that fluid is detected at the second level. In some embodiments, the controller is responsive to the detected ratio of ‘on’ time to total time to control the speed of the first or second pump.

In some embodiments, the first and second fluid level sensors each include one or more electrically conductive electrodes capable of sensing a presence of liquid based on its conductivity. In some embodiments, the first and second fluid level sensors each include two electrically conductive electrodes. In some embodiments, both electrically conductive electrodes of the first fluid level sensor are covered in an insulating material along part of their length.

In some embodiments, one of the two electrically conductive electrodes of the second fluid level sensor is covered in an insulating material along part of its length.

In some embodiments, the controller is configured to receive conductivity signals from each of the four electrically conductive electrodes and, in response, estimate a fluid level in the first or second boiling vessel.

In some embodiments, at least some of the electrically conductive electrodes have different physical dimensions.

The first and second fluid level sensors may form the first liquid sensor or the second liquid sensor.

According to a second aspect, the invention provides a method of treating a liquid, the method comprising the steps:
boiling the liquid in a first boiling vessel using a first heater to generate vapour that includes volatile contaminants;
venting the vapour from the first boiling vessel via a first vapour outlet;
establishing a flow of the liquid out of the first boiling vessel after venting the vapour through the first vapour outlet for a predetermined period;
adding the liquid from the first boiling vessel to a second boiling vessel, and boiling the liquid in the second vessel using a second heater to generate vapour; and,
condensing, in a condenser, the vapour from the second boiling vessel to a liquid that is treated, the condenser having an outlet for outputting flow of the treated liquid.

The first vapour outlet may include a first outlet valve configured open at or above the first pressure. The second boiling vessel may be configured to generate vapour at a second pressure. The second pressure may be greater than ambient pressure and less than the first pressure. The outlet may include an outlet valve that is configured to open at or below the second pressure.

According to a third aspect, the invention provides a multistage treatment device for treating a liquid, the treatment device comprising:
a first boiling vessel comprising a first heater for boiling the liquid in the first boiling vessel to generate vapour including volatile contaminants;
a first vapour outlet configured to vent the vapour from the first boiling vessel;
a liquid outlet for fluid flow of the liquid from the first boiling vessel to a subsequent boiling vessel after a predetermined period of vapour flow through the first vapour outlet;
a second boiling vessel to receive the liquid from the first boiling vessel and comprising a second heater for boiling the liquid in the second boiling vessel to generate vapour; and
a condenser for condensing the vapour from the second boiling vessel back to liquid that is treated, the condenser having a condenser outlet configured to dispense the treated liquid.

In some embodiments, the system includes a controller. The controller is preferably configured to monitor a sensor signal from the second liquid sensor over a period of time to detect splashing of the fluid at a level of the second liquid sensor.

The controller is preferably configured to detect a ratio of ‘on’ time to total time of the second sensor signal, wherein ‘on’ time indicates that fluid is detected at the second level of the second liquid sensor.

In some embodiments, the controller is responsive to the detected ratio of ‘on’ time to total time to control the speed of the first fluid supply device.

In some embodiments, the first and second fluid level sensors each include one or more electrically conductive electrodes capable of sensing a presence of liquid based on its conductivity. In preferred embodiments, the first and second fluid level sensors each include two electrically conductive electrodes. Preferably both electrically conductive electrodes of the first fluid level sensor are covered in an insulating material along part of their length. Preferably one of the two electrically conductive electrodes of the second fluid level sensor is covered in an insulating material along part of its length.

In some embodiments, the controller is configured to receive conductivity signals from each of the four conductivity electrodes and, in response, estimate a fluid level in the boiling vessel.

In some embodiments, at least some of the conductivity electrodes have different physical dimensions.

According to a fourth aspect, the present invention provides a multistage distillation system for a liquid, the system comprising:
a first stage; and
one or more subsequent stages including a final stage;
wherein the first stage comprises a first boiling vessel and a first heater configured to boil the liquid at a first pressure, the first pressure being greater than ambient pressure, such that the boiling liquid generates vapour including volatile contaminants, a first vapour outlet configured to vent the vapour from the first boiling vessel, and a liquid outlet for fluid flow to one of the one or more subsequent stages after a predetermined period of flow of vapour through the first vapour outlet; and,
wherein the final stage comprises a second boiling vessel to boil liquid from the first stage, to generate vapour; the system further comprising:
a condenser to receive the vapour from the second boiling vessel and condense the vapour to a liquid that is treated, and
an outlet configured to dispense treated liquid.

The subsequent stage to which fluid flows from the liquid outlet may be the final stage.

According to fifth aspect, the present invention provides a system for preparing a reconstituted medicament for a patient, the system including:
a power input for receiving electrical power from a mains power wall outlet of less than or equal to 20 amps;
a fluid inlet for receiving untreated fluid;
a fluid treatment device for processing the untreated fluid to produce a treated fluid; and
an outlet for outputting the treated fluid, the outlet including a first coupling formation of a coupling system;
wherein the first coupling formation is adapted to couple with a complementary second coupling formation in fluid communication with a container of concentrated medicament such that, when the first and second coupling formations are in engagement, the fluid treatment device delivers a measured amount of treated fluid to the container to mix with the concentrated medicament and generate a reconstituted medicament suitable for administering to the patient.

In some embodiments, the first and second coupling formations are able to be moved between:
a first open position in which the first and second coupling formations are not touching but held within an at least partially enclosed space in close proximity; and
a second engaged position in which the first and second coupling formations are in sealing engagement to form an internal fluid channel.

In some embodiments, the first and second coupling formations are moved between the first and second positions by a mechanical actuator device. The mechanical actuator device may be automatically electrically controllable. The mechanical actuator device may also be user operated.

Preferably, the system is configured for use in a domestic environment. The system may be powered by mains power of less than 3.2 KW. The system ay alternatively be powered by a solar powered source.

The fluid treatment device is preferably a distiller, the untreated fluid is preferably water and the treated fluid is preferably distilled water.

In accordance with a sixth aspect, the present invention provides a coupling device for coupling a treated fluid from a source to a patient device, the device including:
a first mounting portion adapted to receive an outlet from the source in fluid communication with a first coupling formation;
a second mounting portion adapted to releasably engage with a connector of the patient device having a second coupling formation;
an actuation mechanism configured to linearly slideably move the first or second mounting portion between a first open position in which the first and second coupling formations are not touching but held within an at least partially enclosed space in close proximity and a second engaged position in which the first and second coupling formations are in sealing engagement to form an internal fluid channel.

The patient device preferably includes a container of concentrated medicament. The source may be a fluid treatment device. The fluid treatment device may be a distiller.

The fluid treatment device is preferably adapted to provide the treated fluid as steam and wherein, in the first position, the connector of the patient device is exposed to steam from the outlet to sterilise the connector.

The first mounting portion is preferably fixed and the second mounting portion is linearly slideably moveable relative to the first mounting portion.

The actuation mechanism may include an actuator that is manually actuated by a user. Alternatively, the actuation mechanism may include an electromechanical actuator that is responsive to a control signal.

In accordance with a seventh aspect, the present invention provides a system for filling a container of concentrated dialysate with purified water, the system including:
a water purifying device for purifying water from a source of untreated water; and
a coupling system configured to couple water from an outlet of the water purifying device to an inlet of the container to fill the container and produce a reconstituted dialysate solution.

In some embodiments, the system includes a system controller configured to control a rate of purified water flowing into the container from the water purifying device.

In some embodiments, the coupling system is responsive to the controller to control the automatic coupling or decoupling of the outlet of the water purifying device with the inlet of the container.

In some embodiments, the water purifying device is a distiller. In some embodiments, the controller controls the distiller to produce steam at the outlet so as to perform a sterilising process in the coupling system.

Embodiments of the present invention provide a preparation system to reconstitute a concentrate consisting of a pre-sterilised medicament container and a domestic scale water purification system configured to automatically complete the coupling, filling and decoupling process and further configured such that the decoupling process is arranged to ensure that a predetermined amount of fluid is added to the container without compromising the sterility of said container.

The system may include an inlet for unprocessed water (example water from a domestic plumbing system—mains water).

The purification system may include a water ultra-filter, a reverse osmosis system, a de-ionisation system, a distiller or a combination of the foregoing.

An outlet system may be provided for the processed water, characterised by the inclusion of a coupling device. The coupling device may be configured such that it can couple to a suitable terminally sterilised medicament container, and effect such a coupling in a manner that maintains sterility within the container.

A control system may be provided which can verify the container meets predetermined requirements and ensures that a pre-cleaning process is applied prior to connection. A connection is established for fluid dispensing, and a decoupling process arranged to ensure that flow is stopped when the predetermined volume of fluid is dispensed.

A filtration system, reverse osmosis or similar system are usually operated at ambient temperatures and present a large surface area on the “clean” process outlet side. On startup, special care is required to ensure that these surfaces, and downstream couplings are sterile before commencement of a reconstitution process.

The inventor has realised that a distiller can be configured to operate with the condenser operating at reduced capacity on startup, thus delivering hot water or steam or pressurised steam to a pre-clean region including all downstream surfaces, thus facilitating cleaning on startup without significant additional components.

The receiving container may be delivered with a closure over the connecting means to the container, such as a cap. On removal of this closure, there is a risk of contamination. The precleaning step above is further extended by ensuring that the surfaces thus exposed are included in the pre-cleaning region.

Optional features described above in relation to any aspect or aspects of the invention may, where applicable, also be features of any other aspect or aspects of the invention.

### BRIEF DESCRIPTION OF THE DRAWINGS

Preferred embodiments of the present invention will now be described by way of example only with reference to the accompanying drawings in which:

FIG. 1 is a schematic diagram of a system and device for multistage treatment of a liquid according to a first embodiment of the present invention;

FIG. 2 is a front perspective view of a system and device for multistage treatment of a liquid according to a second embodiment of the present invention;

FIG. 3 is a rear perspective view of the system of FIG. 2 showing a heat exchanger in the inset;

FIG. 4 is a front window view of a degas module of the system of FIGS. 2 and 3;

FIG. 5 is a front window view of an evaporator module of the system of FIGS. 2 and 3;

FIG. 6 a side perspective cutaway view of the degas module of FIG. 4;

FIG. 7 is a side perspective cutaway view of an underside of the evaporator module of FIG. 5;

FIG. 8 is a schematic diagram of a system and device for multistage treatment of a liquid according to a third embodiment of the present invention;

FIG. 9 is a side perspective view of a device for multistage treatment of a liquid according to a third embodiment;

FIG. 10 is another side perspective view of the device of FIG. 9 showing a dialysate bag connected to the device and supported on a weight sensor;

FIG. 11 is a front window view of the device of FIGS. 9 and 10 with the cover removed to show the primary components of the device;

FIG. 12 is a close-up side perspective view of the device of FIGS. 9 to 11 more clearly showing a fluid connection between the device and a dialysate bag;

FIG. 13 is a side perspective view of a load cell weight sensor of the device of FIGS. 9 to 12;

FIG. 14 is side cutaway view of a sterile coupling device for coupling a treated fluid outlet of a fluid treatment device to a connector of a patient device such as a dialysate bag;

FIG. 15 is a top view of the coupling device of FIG. 14;

FIG. 16 is a perspective view of a connector for connecting a patient device to the coupling device of FIGS. 14 and 15;

FIG. 17 is a cutaway view of the connector of FIG. 16; and

FIG. 18 is a schematic diagram of a system for filling a dialysate bag containing concentrated dialysate.

### DETAILED DESCRIPTION OF THE PREFERRED EMBODIMENTS

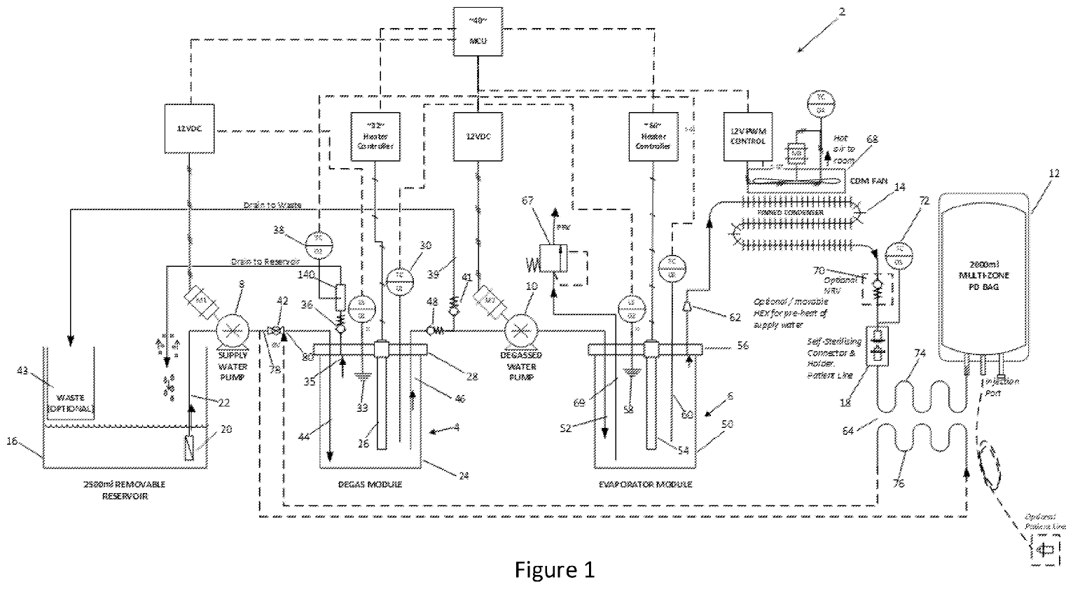

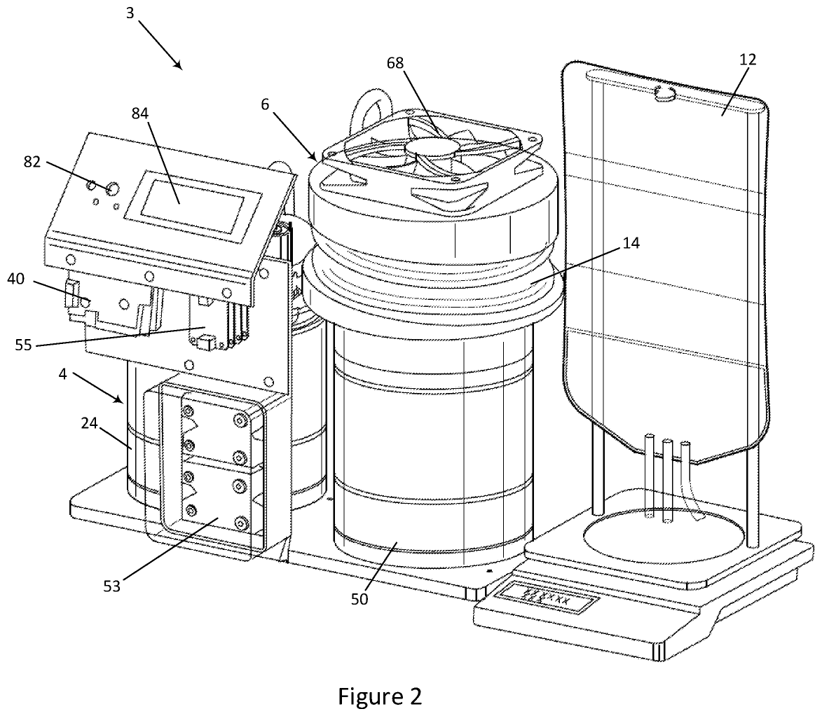

The features and operation of the present invention will initially be described with reference to FIGS. 1 to 7. FIG. 1 represents a first embodiment water treatment device 2, while FIGS. 2-7 represent a second embodiment water treatment device 3. Devices 2 and 3 operate in an equivalent manner with many like features. In the following description and accompanying drawings, corresponding features of devices 2 and 3 are designated with like reference numerals for clarity and simplicity.

Water treatment devices 2 and 3 are two-stage treatment systems for purifying water from a water supply tank 16. While the treatment devices 2 and 3 use a two-stage treatment system (in which the second stage includes distillation), skilled workers will appreciate that additional stages may be incorporated between the first stage 4 and the final stage 6 to increase the stages in the multistage system. By way of example, additional filtering or degassing stages may be added. However, for the purposes of purifying water to a purity suitable for use in a peritoneal dialysis treatment, a two-stage treatment process is typically adequate.

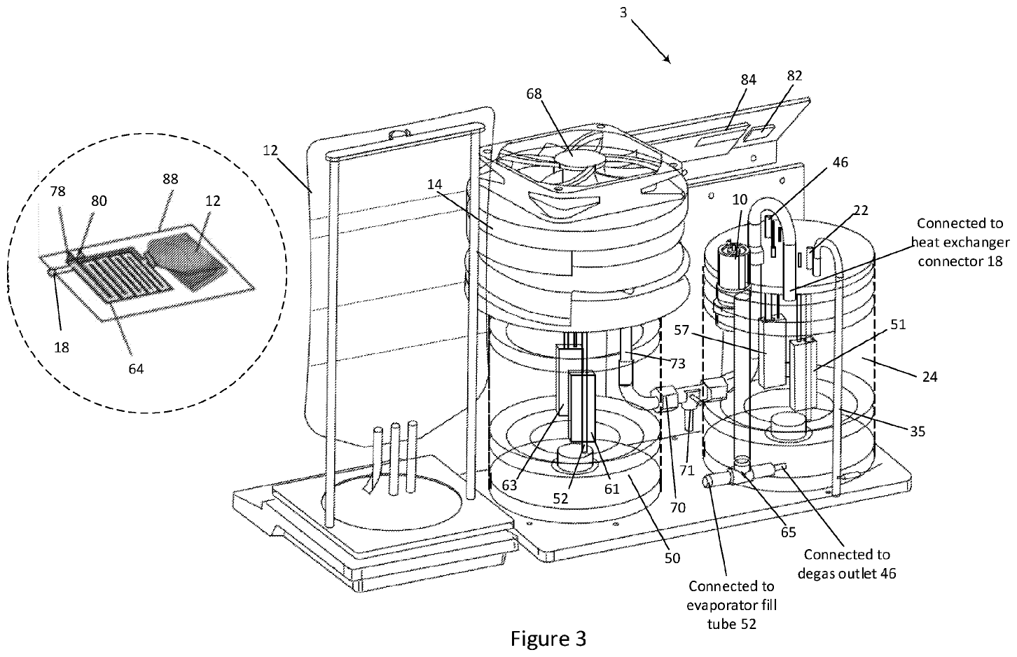

FIG. 1 shows a schematic diagram of the water treatment device 2 being used to fill a dialysate bag 12 for a peritoneal dialysis patient (see FIGS. 37 to 44). FIGS. 2 and 3 show perspective views of the main components of water treatment device 3 set up to fill dialysate bag 12. FIGS. 2 and 3 omit connection hoses that connect the fluid between the different modules and the water supply. FIG. 2 also omits a heat exchanger (described below) that would be connected between the dialysate bag 12 and device 3. However, this is illustrated in the inset of FIG. 3.

The systems use a continuous flow process rather than a batch heating process to deliver the treated water to the sterilising connection 18 with the dialysate bag 12 at a rate of about 1.11 kilograms per hour. The term ‘continuous’ is not restricted to an output flowrate that does not vary, or periodically stop and start, but includes any system in which the liquid (such as water) is being output as treated liquid concurrently while supply liquid is being processed in earlier stages.

The first stage of the treatment system is the degas module 4, best shown in FIGS. 1, 4 and 6. The degas module 4 has a first boiling vessel 24 enclosed by a first sealed top 28. In treatment device 2 of FIG. 1, the sealed top 28 provides a single structure holding all the operational componentry, i.e. a first heater 26, a first water level sensor 34, a first fill tube 44 and a first outlet tube 46 and a vapour outlet 35. Having all the componentry held by the top 28 allows the boiling vessel 24 to be easily removed and descaled (cleaned) during routine maintenance. In some embodiments, boiling vessel 24 is releasably engageable from top 28 by a screw thread or other engagement means such as one or more clips.

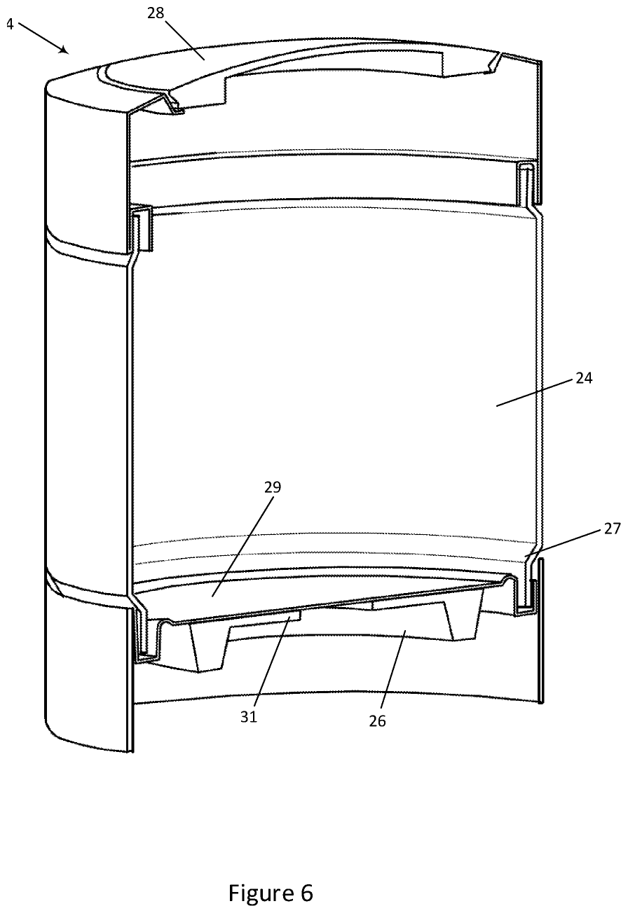

In the degas module 4, water is drawn from a water supply tank 16 or mains water by a fluid control device in the form of pump 8 and delivered to the first boiling vessel 24 by first fill tube 44. In some embodiments, where the system is connected to mains water, pump 8 may not be required and may be replaced by an alternative fluid supply device such as a solenoid valve. The primary requirement of the fluid control device is to be able to control a flow of water into degas module 4. As the water level in first boiling vessel 24 rises, a level sensor 33 detects the water and activates the first heater 26 to heat the water. Although illustrated as a cylindrical heating element in FIG. 1, first heater 26 is preferably implemented as an underfloor heater as will be described below and illustrated in FIG. 6. As the water is heated, steam and volatile gases are expelled from first boiling vessel 24 through vapour outlet 35. When the temperature of the water in first boiling vessel 24 reaches a predefined temperature, water is pumped into the final stage evaporator module 6. This process is described in more detail below.

As illustrated in FIG. 6, in degas module 4 of device 3, the first heater 26 is disposed within a base 27 of first boiling vessel 24. The water in first boiling vessel 24 is in contact with a stainless steel plate 29, which defines an upper surface of base 27. A heat diffuser 31 is disposed underneath plate 29 and in thermal contact therewith. In some embodiments, heat diffuser 31 is formed of a 2 mm aluminium plate, brazed to the underside of plate 29. The first heater 26 includes a curved tubular heating element formed of aluminium packed with electrical insulator/thermal conductor material (e.g. MgO) surrounding a centrally disposed coil of NiCrFeAl acting as the heat source. Heat from the heating element 26 is transferred through diffuser 31 to stainless steel plate 29 to heat water in the first boiling vessel 24. Plate 29 is extended to accommodate embedded safety temperature sensors and electrical connections (not shown).

First heater 26, as well as various other electrical components described below, are able to collectively be powered by a connection to a standard mains power outlet. By way of example, devices 2 and 3 may be powered by a single phase mains supply of 220 V to 240 V at 50 Hz or 120 V at 60 Hz.

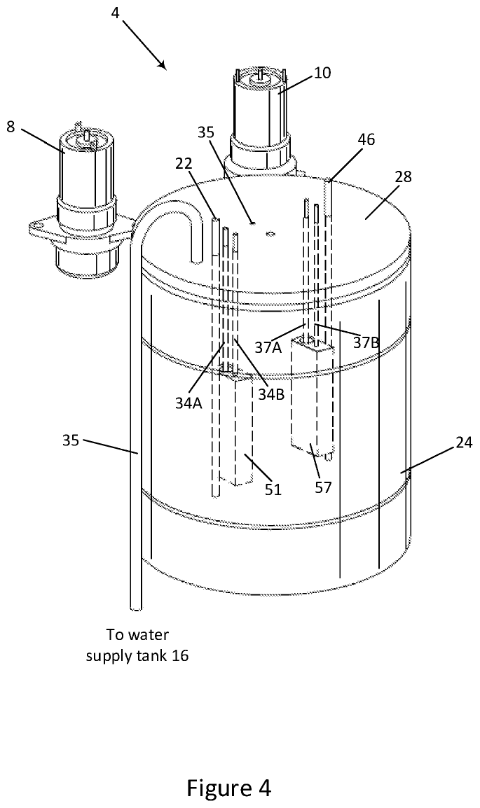

As shown in FIGS. 1 and 4, the degas module 4 has a vapour outlet 35 and a non-return/pressure-reducing steam valve 36, allowing steam and contaminants to vent from the first boiling vessel 24 once it reaches a threshold pressure. Because the pressure within the first boiling vessel 24 is higher than ambient pressure, the temperature of the steam is greater than 100° C. (the normal boiling point of water at atmospheric pressure). In light of this, the first heater 26 provides a direct heat source to directly heat the water in the degas module to about 104° C. to induce boiling.

During this degas stage, the heater 26 uses about 100 W to 160 W to bring the water to boil. More particularly, the heater may use about 120 W to 130 W plus an additional 1.5% of total system power (or approximately 10 W to 15 W) to generate the slightly excess steam flow. The first steam valve 36 operates as a non-return valve as well as a pressure-reducing valve. The temperature of the steam conduit defining vapour outlet 35 downstream of the first steam valve 36 is monitored by the second temperature sensor 38. Sensor 38 measures the surface of the conduit defining outlet 35. This conduit is heated on its inside by about 15 W of condensing steam, and on the outside by ambient air. The conduit temperature measured by temperature sensor 38 is the balance between both, which becomes a measure of whether the 15 W (target outflow) is being achieved.

The first temperature sensor 30, being a vapour sensor monitors the temperature of the steam conduit of vapour outlet 35 at the top of the first boiling vessel 24. The second temperature sensor 38 is used to control the temperature within the first boiling vessel 24 by balancing heat transfer from the interior of the degas module 4 to the exterior of the degas module 4.

vapour outlet 35 provides a steam outlet for outputting steam within first boiling vessel 24. Gas contaminants get entrapped in gas bubbles and will be carried out of the system via vapour outlet 35. Vapour outlet 35 includes a heat exchanger 140 which acts to transfer energy from the heated vapour exiting outlet 35 to the cooler ambient atmosphere. In traversing heat exchanger 140, the steam is rapidly cooled and condenses back to a liquid. As shown in FIG. 1, vapour outlet 35 is positioned to deposit the condensed water back to water supply tank 16. This avoids the need for the user to provide an additional fluid collection means. At the same time, contaminant gases that exit via first outlet tube 46 with the steam remain as gases and are carried away out of the system completely.

The first steam valve 36 is conveniently provided as a spring valve in which a ball or disc is biased into sealing engagement with the valve seat. However, skilled workers will readily understand that other non-return valve constructions would be suitable. The non-return function of the first steam valve 36 ensures that no external gases can be drawn back into the first boiling vessel 24 to contaminate the water. As a pressure-reducing valve, the first steam valve 36 maintains the steam at the top of the boiling vessel 24 at an elevated pressure. For example, a pressure increase of 7.43 kPa will increase the boiling temperature of the water in the degas module 4 by about 2° K.

It will be appreciated that first steam valve 36 is not essential in some applications such as in domestic devices. However, in medical grade devices, first steam valve 36 may be essential to prevent external gases and contaminants being drawn into the device via first outlet tube 46 when the device is not powered.

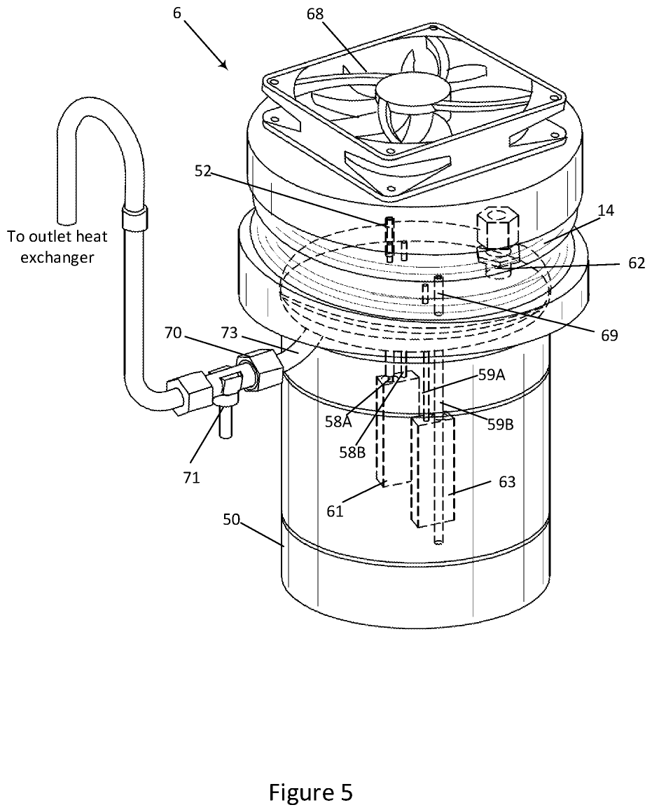

First water level sensor 33 and second water level sensor 58 may be a type selected from a variety of known level sensors, such as mechanical, optical or ultrasonic level sensors. As shown in FIG. 4, first water level sensor 33 comprises two pairs of electrically conductive electrodes 34A and 34B and 37A and 37B. Similarly, in FIG. 5, second water level sensor 58 comprises two pairs of electrically conductive electrodes 58A and 58B and 59A and 59B. Each conductive electrode pair are exposed to the water in the respective boiling vessel 24 and 50. A low-voltage, current-limited power source is applied across the electrodes to sense conductivity. When the electrodes are calibrated, a measure of high conductivity indicates that water is present, while low conductivity indicates the water is not present at the level of the electrode (and hence highly resistive air is detected). The electrodes within each boiling vessel are set at different heights based on the level change of water during the boiling process. The operation of first and second level sensors 34 and 58 are described below. In other embodiments, conductive electrodes 34A, 34B, 37A, 37B, 58A, 58B, 59A and 59B can be replaced by through-wall sensors that are housed outside the sidewalls of boiling vessel 24 and which can sense internal water level using a capacitive or optical sensor in a similar manner to that of the electrodes.

The first stage of the treatment (degassing) removes gas contaminants from the water but is unable to remove particulate contaminates. These are passed to the second stage for removal as described below.

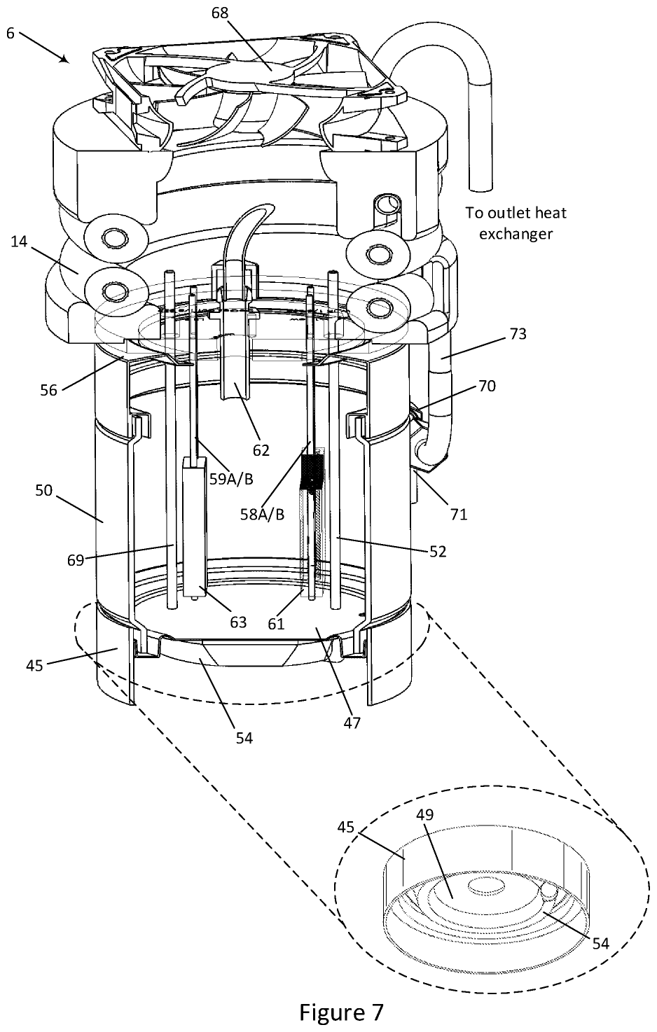

The second and final treatment stage involves distillation and occurs in the evaporator module 6. The evaporator module 6 has a second boiling vessel 50 with a second sealed top 56. Like the degas module 4, the second sealed top 56 provides a mounting structure for the second fill tube 52, the second water level sensor 58, the second heater 54, a second temperature sensor 60 and the second steam outlet 62. Although illustrated as a cylindrical heating element in FIG. 1, second heater 54 is preferably implemented as an underfloor heater as will be described below and illustrated in FIG. 7.

Referring to FIG. 7, in device 3 the second heater 54 is disposed within a base 45 of second boiling vessel 50. Like with the degas module 4, the water in second boiling vessel 50 is in contact with a stainless steel plate 47, which defines an upper surface of base 45. A heat diffuser 49 is disposed underneath plate 47 and in thermal contact therewith. The heat diffuser 49 may be formed of a 2 mm aluminium plate and brazed to the underside of plate 47. The second heater 54 includes a curved tubular heating element similar to that of first heater 26 described above. The heating element transfers heat through heat diffuser 49 to the stainless steel plate 47 to heat water in the second boiling vessel 50. Plate 47 may be extended to accommodate embedded safety temperature sensors and electrical connections (not shown).

Degassed water from the degas module 4 is drawn from the first boiling vessel 24 through the degas water outlet valve 48 by the second pump 10 and into the second fill tube 52. Degassed water partially fills the second boiling vessel 50 and second heater 54 provides a direct heat source to boil the water to generate steam at a rate that provides a flow rate of recondensed treated water to the self-sterilising coupling 18 of about 1.11 kilograms per hour. The steam generated in the second boiling vessel 50 flows through steam outlet 62 into a condenser 14 with a series of cooling fins. The condenser 14 is within an airflow from a cooling fan 68. The steam recondenses to water in the condenser and exits the condenser outlet valve 70 via a condenser outlet tube 73.

Referring again to FIG. 1, treated water from the outlet valve 70 flows to the coupling 18 for fluid connection to the dialysate bag 12. Between the fluid coupling 18 and the dialysate bag 12 is a heat exchanger 64, illustrated in the inset of FIG. 3. Here the relatively hot recondensed water is further cooled to approximately ambient temperature, and flows into the dialysis bag 12. Likewise, the relatively cool supply water drawn from the supply tank 16 is heated immediately prior to being fed into the first fill tube 44. Skilled workers will appreciate that this reduces the input energy required to boil water in the degas module 4.

As shown in FIG. 3, a drain-off outlet 71 and associated valve is included in the condenser outlet tube 73 to provide capability to sample the water quality in the condensed and distilled liquid before being input to a dialysate bag. Drain-off outlet 71 may include a tap or valve that can be switched on or off.

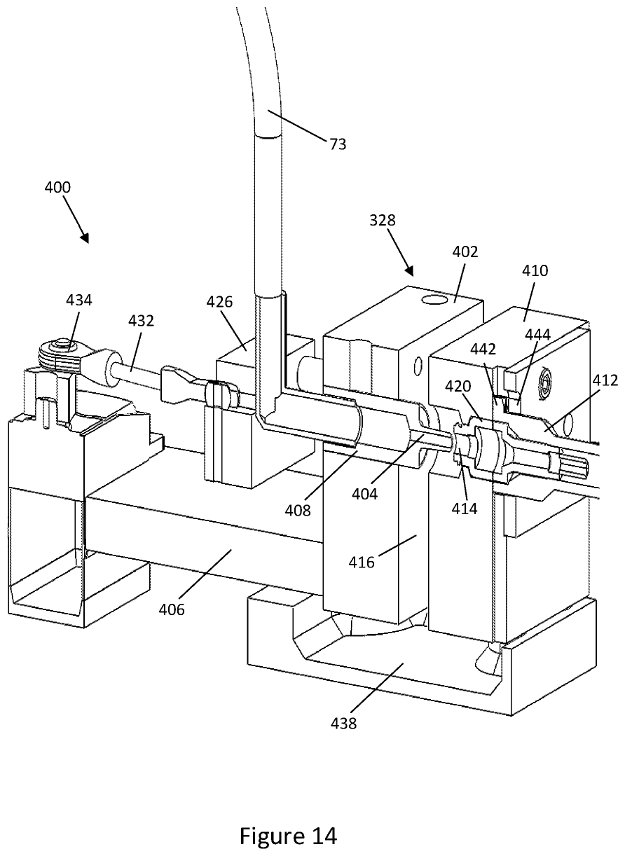

The condenser outlet valve 70 is a non-return valve such that any external contaminants are kept out of the condenser and the evaporator module. Outlet valve 70 is configured to open when the pressure in condenser 14 reaches a predefined threshold pressure sufficient to open the valve. Referring to FIG. 1, a fourth temperature sensor 72 is provided immediately upstream of the self-sterilising, coaxial connector 18 (described below) to detect the inner surface temperature of the conduits at the fluid connection. The sealed fluid connection 18 couples together in two stages. In stage 1, fluid communication from the condenser side coupling to the unsealed surfaces on the bag side coupling is established for sterilization by steam. Then in stage 2, the bag side coupling is pushed into full engagement with the condenser side coupling. This self-sterilising coupling process is described in greater detail below with reference to FIGS. 14 to 17.

As described, embodiments of the present invention use steam as a mechanism to remove gases and contaminants from the system. Separate heaters in the first and second stages allow adjustment of a level of steam to remove gases. This control is performed by master control unit 40 in conjunction with heater controllers 32 and 66, and provides for controlling the amount of power to satisfy the process heating needs as well as providing for parasitic heat losses from the system.

Both the first heater 26 and second heater 54 are disposed within the respective boiling vessels 24 and 50 or in the floor of the boiling vessels 24 and 50 in contact with the water to provide direct heat sources. This improves the efficiency of the device and allows tighter control over the input heat energy. Thus, no external heat sources such as steam inputs are required. Degas module 4 and evaporator module 6 define separate independently controlled environments within the system.

With reference to FIG. 2, master control unit 40 includes a microprocessor connected to an LCD display 84 (or indicator panel in device 2) for displaying information to a user of device 3 and an on/off button 82. Master control unit 40 is also connected to a power supply and distribution unit 53 that is connected to mains power and configured to distribute power to the various components of device 3. Master control unit 40 is further connected to conductivity sensors 55 which are connected to respective ones of conductivity electrodes 34A, 34B, 37A and 37B, 58A and 58B and 59A and 59B.

The operation of the device will now be described in stages of the treatment process. Throughout the description, exemplary values will be used for temperatures, flow rates, power levels and other parameters. It will be appreciated that these are for illustration only and will depend on the specific system design and scale size. For example, in countries with lower voltage mains supply (100-120 V), power will be more restricted (<1,200 W to 1,500 W) than in regions with 220-240 V 2200-3,000 W).

### Degas Module Start-Up Sequence

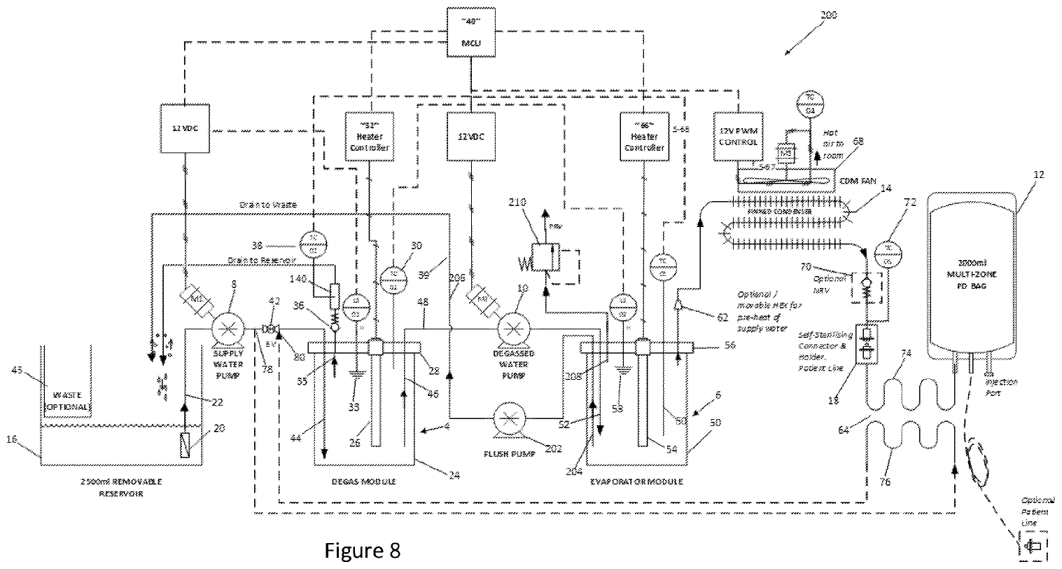

Referring to FIGS. 1 to 36, the user switches on the device using the on/off switch 82 (see FIGS. 2 and 8) and the master control unit 40, activates the first peristaltic pump 8, which draws water from the supply tank 16 through inlet filter 20, and inlet tube 22 to the degas module inlet valve 42. The inlet valve 42 is opened and the supply water begins filling the first boiling vessel 24 via the first fill tube 44. When the first water level sensor 33 indicates sufficient water in the first boiling vessel 24 (i.e. the first heater element is adequately immersed), the first heater 26 is activated by heater controller 32 to bring the water to boiling temperature. As discussed above, the degas module is sealed such that the interior pressure is greater than ambient pressure. Preferably, the degas module includes non-return steam valve 36 that is configured such that the internal pressure elevates the boiling temperature of the water to be between 101° C. to 105° C.

As best shown in FIG. 1, as the internal pressure of the first boiling vessel builds, the first steam valve 36 reaches the threshold pressure, and opens to vent steam through the vapour outlet 35. In some embodiments, steam valve 36 is not included and steam is allowed to exit through outlet 35 at any pressure. The steam condenses quickly in the tube of vapour outlet 35 and the liquid is returned to water supply tank 16. The entrained gas contaminants exit the vapour outlet 35 as gases and are exited from the system. The outflow through the outlet 35 is intended to be a low steam flowrate of approximately 1.8 litres per minute to 2.2 litres per minute. If power to the first heater 26 is set to maximum (typically between 800 W to 1,100 W), the flowrate of the steam is approximately 50 litres per minute. This is excessive and so the output from the first temperature sensor 30 is used to reduce and condition the power input to the degas heater 26 when appropriate.

As boiling initiates, the surface of the heater 26 starts producing steam locally when the bulk water temperature is still below the boiling point. The first heater 26 output is reduced (for example, at 80° C. heater 26 operates at 100% output while at 100° C. to 105° C., the heater controller 32 reduces the power back to 70-350 W or approximately 15% output).

The steam/gas outflow through the outlet 35 is measured by the second temperature sensor 38, which is located within heat exchanger 140 and positioned to measure an average temperature within the heat exchanger (e.g. half way between ambient temperature and steam condensing temperature). Within heat exchanger 140,, the temperature is between ambient (approximately 20° C.) at the downstream end and the temperature within the first boiling vessel 24 (approximately 102° C.) at the end proximal to outlet 35. If ambient temperature is 20° C., then temperature sensor 38 is positioned to measure 60° C. when steam outflow is at design point (2 L/min). If the steam flowrate through outlet 35 is zero, the second temperature sensor 38 reading will be close to ambient. Conversely, if the flowrate of steam through the outlet 35 is high, the temperature sensed by the second temperature sensor 38 will be approaching 102° C. (the temperature of the steam inside of the first boiling vessel 24).

By monitoring the steam temperature at the first steam valve 36, the temperature sensor output becomes a proxy measure for the steam flowrate through the valve. This provides ongoing evidence that steam, and therefore entrained volatile gases are being ejected from the system. Furthermore, because the boiling temperature within the first boiling vessel 24 is elevated, any volatile contaminants that may have a boiling temperature at or slightly above 100° C., are more thoroughly eliminated before transferring to the evaporation module 6.

Referring to FIG. 3, to enhance the degas process, in some embodiments, one or more ultrasonic transducers (not shown) are disposed within a Tee section 65 of first outlet tube 46. The Tee section 65 includes a vertically extending pipe, which is connected to a second gas outlet 39 (see FIG. 1), for expelling gas bubbles. In other embodiments, one or more ultrasonic transducers are disposed within or on boiling vessel 24. These transducers emit ultrasonic waves to the water within the boiling vessel 24 or within outlet tube 46 and aid the degas process by agitating the water and agglomerating smaller gas bubbles into larger bubbles. These larger gas bubbles rise to the surface of the liquid more easily than small bubbles and escape more efficiently through vapour outlet 35 or via the second gas outlet 39 in outlet tube 46.

As shown in FIG. 1, second outlet 39 includes a corresponding non-return valve 41 for restricting airflow back into the system at this stage. Gases and steam removed from the system via outlet tube 46 are output to a waste tank 43 during the initial degas start-up sequence. In some embodiments, second outlet 39 is located after second pump 10, as pump pressure can further assist the flow.

### Evaporator Module Start-Up Sequence

When the second temperature sensor 38 indicates that steam and other volatile gases have been ejected from the first boiling vessel 24 for a predetermined period (e.g. 30 seconds or 1 minute), the second peristaltic pump 10 can be activated. The internal pressure in the first boiling vessel 24 may be higher than that of the second boiling vessel 50 and therefore the second pump 10 is more accurately described as a flow control device.

The skilled person will understand that, in this context, the term “predetermined” means that the period of time is of a duration that is defined or pre-set in advance of operation of the treatment system. The duration of the period of time may be unalterable, e.g. fixed in the hardware of the treatment system. The duration of the period of time may be configurable in advance of operation of the treatment system, e.g. selectable or tuneable, e.g. by a user or maintenance person via an input provided on or for the treatment system or via an input from an external controller. The treatment system may automatically select or configure the duration of the period of time prior to operation, e.g. based on input data or settings.

While the flowrate of degassed water out of the second fill tube 52 into the second boiling vessel 50 would be higher than the “normal” flowrate of 1.11 kilograms per hour specified for filling the dialysate bag, typically the second boiling vessel 50 has an internal volume of approximately 200 millilitres and a fill time of around 5 minutes via an inlet flowrate of around 2.4 kilograms per hour. At this higher flowrate, the power requirements of the first heater 26 significantly increases. For example, to achieve a nominal flowrate of 1.11 kilograms per hour at the fluid coupling 18, the activation power to the first heater 26 needs to increase by 250 W to 300 W. This power requirement is comfortably provided by a device running on a domestic power outlet. Thus, the power to the first heater 26 may be increased to decrease the fill time of the evaporator module (e.g. the fill time associated with 250 W of additional heater power is less than that of the nominal flowrate).

Similarly, the speed of the second peristaltic pump 10 is controlled inversely by the second temperature sensor 38 to ensure there is positive steam/gas flow to control a maximum rate of degas water removed from the degas module.

As discussed in more detail below, the first pump 8 is at least partially controlled by the first water level sensor 33.

Cascade control systems such as this are often prone to instability, resulting in flow surges and non-linear behaviour despite some level of feedback control. These periods of instability introduce contamination risk if the speed of the first pump 8 and the second pump 10 are higher than nominal, even if just momentarily. The first heater may not maintain the intended temperature within the first boiling vessel 24. While these instabilities are merely transitory, it creates a risk of non-degassed water passing from the first boiling vessel 24 to the second boiling vessel 50. To address this, the treatment system 2 uses feedback control with inherent compensation for large short-term fluctuations. In particular, the water level in small vessels such as the first boiling vessel 24 and the second boiling vessel 50 will vary significantly when boiling. In light of this, the first water level sensor 33 and the second level sensor 58 should maintain a smooth or damped output signal that is indicative of a mean water level rather than instantaneous level. To achieve this, in some embodiments, the first water level sensor 33 and second water level sensor 58 is provided in the form of electrical capacitors.

By providing the first and second water level sensors (33 and 58) in the form of capacitors, their output is not overly sensitive to the constant fluctuations in the water surface caused by boiling. Instead, the capacitive sensor has electrodes spaced apart such that the intervening dielectric is partially water. The dielectric constant of air differs from that of water and as the water level rises or falls, the combined dielectric constant (the proportion of air dielectric and the proportion of water dielectric) will vary. This in turn varies the capacitance of the sensor and the output signal becomes indicative of the mean water level. However, the changes to capacitance are not instantly sensitive to any minor fluctuations in the surface of the water caused by the turbulence of boiling. This is because the capacitor is sensitive to the water inside a tube, and the water inflowing is restricted, and less prone to the splashing outside the tube. In this way, the outputs from water level sensors 33 and 58 also provide useful feedback control to the master control unit 40 to operate the first and second pumps 8 and 10.

This control is described in more detail in the next section.

### Heater and Pump Control Using Level Sensing

Strict control of first heater 26 is required for safe and efficient operation of device 2. In device 3, the first heater 26 is located within base 27 of first boiling vessel 24 and immersion of first heater 26 is generally straightforward (a fluid level of about 20 mm is typically sufficient to cover an “underfloor” style heater). However, in device 2, first heater element 26 is extended vertically within first boiling vessel 24 and full immersion takes time. If the water does not cover the entire surface area of the heater element, the energy flow will rapidly increase the temperature of the heater itself resulting in a dangerous overheating situation. This is managed in the present invention using level sensor 33 within first boiling vessel 24, which disables first heater 26 if the water level falls below a safe lower limit.

In some embodiments, a “dry-switch-on” protector (as required by Standard IEC60601) may be included in thermal communication with a surface of first heater 26. This dry-switch-on protector may be operable in the event of a system malfunction such as faulty level sensor 33. In some embodiments, a second level “dry-switch-on” protector or thermal fuse may also be provided as additionally required by the fault tolerance requirements of IEC60601.

As such, level sensor 33 has two main requirements: to control the pump flow (via both first pump 8 and second pump 10); and protect the heater.

As the water is boiling in first boiling vessel 24, the surface agitation causes the effective surface of the water to rise. In the case of a small boiling vessel with a surface energy in the region of 15 W/cm², the inventor has identified that agitation increases the effective surface height by about 12-17 mm. On this basis, an example control sequence implemented by master control unit 40 might be:
i. Activate first pump 8 until 20 mm of water is detected by level sensor 33.
ii. Switch first pump 8 off, activate first heater 26. As the fluid boils, the sensor “sees” a level of 20+15=35 mm.
iii. Boiling and processing continues, and the sensed boiling fluid level eventually reduces to the sensor setting of 20 mm. Because level sensor 33 now signals “low water”, the first heater 26 is powered off, while the first pump 8 is activated. However, as boiling now stops, the true level of the fluid is only 5 mm, and there will be a delay as the first pump 8 restores the level from 5 mm to 20 mm and the first heater 26 re-starts.

In this sequence, when the first heater 26 is deactivated, the system is in a “dangerous” condition—degassing is not effective, and, a partial vacuum can occur risking ambient air being drawn into the system, compromising sterility and purity.

Embodiments of the present invention therefore include a level sensing system comprising two pairs of conductivity sensing electrodes 34A and 34B, and 37A and 37B, as best shown in FIG. 4 and described above. The electrodes may be disposed within boiling vessel 24 as shown in FIG. 4 or may be disposed outside of boiling vessel 24 as through-wall sensors. The electrode pairs 34A and 34B are set at a height difference that is greater than the level-change occasioned by the boiling process (in the illustrated case, more than 12-17 mm, typically 20 mm). Pairs of electrodes are used for reliability and redundancy purposes. However, it will be appreciated that single sets of electrodes may be used in an equivalent control process.

A first lower electrode pair 34A and 34B is set at a first height of about 10 mm and a second upper electrode pair 37A and 37B is set at a second height of about 30 mm. The master control unit 40 is configured such that the operation of first heater 26 is sensitive only to the lower electrode pair 34A and 34B, whereas the control of pump 8 is sensitive only to the upper electrode 34B.

Using the level sensing electrodes described above, a control sequence like the following may be implemented by master control unit 40 (from an empty start). This control sequence may be implemented with a pair of single electrodes at different heights but the significance of two pairs of electrodes will become apparent below. A corresponding control sequence can be implemented in the evaporator module 6 using electrodes 58A and 58B (as one electrode or separately) and 59A and 59B (as one electrode or separately), second pump 10 and second heater 54.
i. The first pump 8 is configured to operate at a highest flowrate, for example 300 ml/minute until electrode pair 34A and 34B (10 mm) detects fluid.
ii. At this point, the fist heater 26 is switched on. In the case of degas module 4, the first heater power can be approx. 200% to 1,000% of a nominal value as the evaporator module 6 will not yet require power during a “dry” start-up. For example, if the ongoing power requirement is typically 200 W, during this phase the power can be 1,400 W-2,200 W.
iii. The flowrate of first pump 8 is then reduced to a value just above the operating requirement, and therefore the fluid level continues to slowly rise from 10 mm to approach the 30 mm electrode pair 37A and 37B.

As the first heater 26 is at high power, and the first pump 8 is at reduced speed, as the water boils, the agitation effect will quickly modify the effective level as seen by the higher electrode pair 37A and 37B. As such, while the “true” fluid level is only 15 mm, the 30 mm electrode pair 37A and 37B will begin to detect the presence of fluid.

The inventor has identified that the sensed “reading” from the upper electrode pair 37A and 37B is, initially quite intermittent, as splashing from the boiling surface triggers a signal, and then a few milliseconds later, the “signal” is gone. The inventor has found that if this signal from the upper electrodes is monitored over a 30 second time period, master control unit 40 can be used to count the ratio of “on” time (being time when the upper electrode pair 37A and 37B detect fluid) to total time of the upper electrode signal. This figure can be shown to be reasonably consistent as the “true” fluid level rises, varying smoothly from “always off” to “always on”. This enables a closed loop feedback system to be deployed. The “setpoint” is a target of say 50% “on”. The actual “on time %” in a given window (typically 20-45 seconds) provides the feedback, and the error between target and feedback is used to adjust the speed of first pump 8.

A simple method where the pump speed is proportional to this error has been found to be effective. i.e. if the “on” time is detected to be 100%, the pump speed is set to zero, and if the “on” time is 0% the pump speed is set to 100%. In practice, better results are achieved by using a Proportional Integral Differential (PID) strategy (see below) rather than P only.

The pump and heater control strategy devised above, using conductive electrodes is sensitive to the conductivity of the water. If very pure water is used as the source, the conductivity can be in the range of 2-5 μS, whereas typical regional tap water can be in the range of 100-800 μS. As the water heats from ambient to boiling, the conductivity increases by 250-300%. In the case of the evaporator module 6, as successive volumes of water are distilled, all the dissolved solids remain in the second boiling vessel 50, and the conductivity, which is mostly proportional to the total dissolved solids (TDS) will quickly increase. If 2 L of pure fluid are produced, and the associated TDS remain in the residual 200 ml in the boiler, the concentration is now 10 times higher, commonly in the region of 8000 μS for cold water, which is equivalent to about 20,000 μS when boiling.

Thus, it is advantageous to devise a strategy for reading a conductive electrode considering a variation in conductivity from 2-20,000 μS, and in particular, when used as a level sensing device as described above, where the system is attempting to detect micro splashes in a millisecond timescale. Small residual traces of surface fluid bridging the electrode mounting surfaces will have little impact at 2 μS, but at 20,000 μS may give a resistance reading lower than the electrode when fully immersed in a 2 μS fluid.

Another problem emerges when contemplating such a method, even when the sensitivity of the electronics can be “tuned” to operate across the full spectrum of readings, as even a small voltage is applied to the electrode, the resulting current flow will eventually result in ionic corrosion of the electrode or the “ground plane” (vessel wall). This can be overcome by reversing the anode/cathode relationship, where the electrode is excited with a voltage switching from positive to negative relative to the vessel body. When using a metal body for the boiler, the requirement to connect to safety ground can often compromise the ability to use as part of an AC sensing circuit.

The inventor has found that using a four electrode design can overcome the above limitations, by being independent of the vessel body, and having reduced sensitivity to variations in conductivity.

First pair of electrodes 34A and 34B are arranged so that the downward depending conductive electrodes are insulated along most of their length by being covered on lateral sides by a coaxial insulating sleeve 51 formed of material such inert plastics or polymers like polycarbonate or polyimides. The partial covering of the electrodes by insulating sleeve 51 leaves only a short distance near the tip of the electrode pair immersed in the fluid (2.4 mm diameter each, 8 mm separation, and with 8 mm immersed). The second pair of electrodes 37A and 37B are of similar geometric construction to the first electrode pair but only one electrode 37B of this second pair is insulated along most of its length (being more or less identical to one of the first pair) by being surrounded on lateral sides in a coaxial insulating sleeve 53. The other electrode 37A of the second electrode pair is not insulated along its height and is exposed to the water.

Consider the filling scenario, as the water rises to cover the first 8 mm of each electrode pair, the “reading” of resistance between each electrode pair will be more or less the same. If the fluid is 2 μS conductivity, both electrodes 34A and 34B record a high resistance, and equally with very conductive fluid they both record a very low resistance. In fact, the electrodes can be conveniently connected as two arms of a Wheatstone Bridge, where the reading circuit need only be sensitive to the ratio of the reading between both rather than the absolute value.

As the fluid level rises from this first level (detectable by both electrodes 34A and 34B as “safe to turn the heater on”), the rising fluid will cover the insulated portion of electrodes 34A and 34B, and therefore the “reading” will remain fixed whereas the fluid rising along the surface of the other pair makes contact with an increasing surface area, and therefore a falling resistance value. The attached control system can now see a simple ratio between the reading of electrode pair 34 versus electrode pair 37 and use this as a control input to a PID control system operating the pump. The “setpoint” becomes a target ratio of readings of say 1.5:1 up to about 4.0:1 representing a fluid level which is a multiple of the reference electrode immersion distance (8 mm) above the minimum fluid level. (e.g. 12 mm to 32 mm above the minimum level).

The four electrode system described above can beneficially be interrogated electronically using the Wheatstone bridge method as known. The inventor has however found that by carefully selecting the electrode dimensions, each electrode pair can be “read” by an independent signal amplifier as would be commonly known in TDS meter design. Such electrodes, however, cannot cope with the full spectrum of resistance variation from cold degas conditions to boiling evaporation conditions. The inventor has however devised an electrode method to overcome this.

In the evaporator module 6 of the presently described embodiments, the first reference electrode pair 58A and 58B use an exposed length of only 2-3 mm at 8 mm centres. The second electrode pair 59A and 59B require an exposed height of about 40-50 mm to detect fluid levels and are spaced at centres of about 30 mm. At very low (minimum fluid levels), the second electrode pair will give reading lower than the reference pair. The electrode pairs 58A and 58B and 59A and 59B are partially surrounded on lateral sides by respective insulating sleeves 61 and 63, which operate in a similar manner to that described above for the degas module 4.

### Output Heat Exchanger

The treated water output flowrate through the condenser valve 70 is nominally 1.11 kilograms per hour. Through the optional use of the heat exchanger 64, shown in FIGS. 1 and 3, this flowrate can be increased to 1.215kilograms per hour which represents an efficiency increase from 96.9% to 106%. This measure is calculated by arbitrarily defining 100% efficiency based on the energy needed to heat water initially at 20° C. to vaporisation. Heat loss to ambient, fan power and steam loss from the degas module result in an efficiency of 96.7% rather than 100%.

By using heat recovery, the outlet from the condenser at 98° C. is cooled to 43° C., and the cold incoming water at 20° C. is heated “for free” up to 74.8° C., therefore claiming an efficiency of 106%. As discussed above, the heat exchanger 64 preheats the supply water being pumped to the degas module 4 whilst simultaneously cooling the treated water flowing to the dialysate bag 12.

Impurities separate from the supply water in the first and second boiling vessels (24 and 50). As the fluid is heated in the boiling vessels (even before reaching boiling point, many impurities exceed their stability limits and separate from the water). Entrained gases come out of suspension in the water and adhere to the exterior of the first and second heaters (26 and 54). Eventually these bubbles rise to the fluid surface in the boiling vessel.

Other particle suspensions attach to the exterior of the first and second heaters (26 and 54). These deposits are commonly referred to as “scale”. With the build-up of scale deposits, the performance of the heater is significantly impacted. Another concern relates to certain impurities that can pass through the degas module 4 and the evaporation module 6 without being removed and remain in the treated water flowing through the condenser outlet valve 70. These impurities can be targeted and removed using particulate filters or by granular absorption carbon (GAC).

With these factors in mind, there are clear benefits to using a heat exchanger that is insensitive to gas collection and scale build up as well as incorporating a pre-filter (not shown). Likewise, the heat exchanger should be configured for use by relatively unskilled operators such that they can ensure ongoing optimal performance.

In view of these issues, the heat exchanger 64 is preferably a disposable heat exchanger configured as a tube-in-tube design having an inner tube 74 and an outer tube 76, as shown in the inset of FIG. 3. The inner tube 74 is part of the tubing that connects the self-sterilising connection 18 to the dialysate bag 12 to provide a fluid conduit to cool heated water from the condenser 14. The outer tube 76 encompasses the inner tube (not shown in the schematic representation of FIG. 1) and provides a fluid conduit between inlet tube 22 and first fill tube 44 of first boiling vessel 24. The flow of cool water through outer tube 76 acts to cool the heated water flowing in the inner tube 74. Simultaneously, the heated water flowing in inner tube 74 heats the cooled water flowing in outer tube 76. This is described in more detail below.

Surface features on the exterior of the inner tube 74 or the interior of the outer tube 76 (or both) maintain the required spacing between the inner and outer tube walls for sufficient flowrate. The annular gap between the inner and outer tube walls may also be provided with porous media such as fibrous material of the type commonly used in fluid filters. Advantageously, the fibrous filters may be impregnated with GAC particles.

In one embodiment, the inner tube 74 from the self-sterilising connection 18 to the dialysate bag 12 is 86.5 millimetres (at a diameter) PVC tube. The overall length of the tube connecting to the dialysate bag is 1 metre, however, skilled workers will understand that only part of this length will be in the heat exchanger 64.

Even though plastic such as PVC has a low thermal conductivity (typically about 0.19 W/mK), the overall heat transfer coefficient is significantly impacted by the heat transfer coefficient from the fluid (water) to the tube surface. For example, if the PVC tube 74 were replaced by a stainless steel tube (which has almost 85 fold improvement in heat transfer coefficient), the overall heat transfer coefficient of the heat exchanger is improved only 4.5. Given this marginal improvement in heat transfer performance, the use of PVC tube or other low cost polymer may be preferred. The heat exchange performance and filter performance are easily and cost-effectively improved by simply increasing the length of the flow path. Residency time of the fluid (water) through the filter medium is a critical factor as the connection to the dialysate bag 12 is external to the treatment device 2. There is little constraint on tailoring the flow path length through the heat exchanger and the filter medium to optimise both the heat transfer and filtration.

### Output Heat Exchanger Manufacture

The PVC inner tube 74 is configured to define a flow path that provides suitable residency time of the fluid to provide suitable cooling of the heated fluid (and corresponding heating of the incoming cool fluid) for delivery to the dialysate bag 12. The outer tube 76 is provided by two opposing sheets of fibre-coated material. These fibre-coated sheets sandwich the serpentine inner tube 74 such that the fibre coatings are in face-to-face contact. The sheets are then welded to each other along the interstitial gaps between each meander of the serpentine inner tube 74. In effect, this forms a sleeve defining a serpentine flow path corresponding to that of the inner tube 74. This can be illustrated in the inset of FIG. 3.

The sheet material may be configured to connect with each other via detached engagement connection lines. This provides the ability to vary the flow path length through the heat exchanger and water filter. The mutually opposing sheets may also be provided with a ribbed feature along the inner surface as is commonly used in vacuum packing film to ensure a flow path exists from the upstream end to the downstream end of the outer tube 76.

In alternative embodiments, the 1 metre long PVC inner tube 74 is not serpentine but straight and the two opposing sheets are reconfigured as elongate strips welded or otherwise bonded to form a tube around the inner tube 74. Similarly, a further alternative configuration provides a tube-in-tube co-extrusion to provide the inner and outer tubes 74 and 76.

This type of heat exchanger is simple and low cost to manufacture and may be used as a single-use or disposable heat exchanger. This is advantageous for medical use applications.

### Output Heat Exchanger Use

As shown in FIG. 1, the first pump 8 delivers supply water to the degas module inlet valve 42 as well as the upstream end 78 of the heat exchanger outer tube 76. In some embodiments, inlet valve 42 and associated heat exchanger 64 is not needed. The downstream end 80 of the heat exchanger outer tube 76 is on the downstream side of the degas module inlet valve giving users the option of simply bypassing the heat exchanger (perhaps during initial start-up) or direct flow to the heat exchanger (during steady state operation).

Two stub-tubes (not shown) are arranged transverse the longitudinal extent of the PVC inner tube 74 a short distance from the upstream and downstream ends of the heat exchanger provide a connection means into the fluid channel formed by the welding operation. These connections connect to either side of the degas module inlet valve 42 such that the flow through the outer tube 76 is counter to the flow through the inner tube 74.

The fibre coating of the sheets defining outer tube 74 provides a filter material that is conveniently provided as an admixture applied to the outside of the PVC inner tube 74 and/or as a surface treatment on one or both of the mutually opposing sheets forming the outer tube 76.

### Shutdown Procedure and Flushing

Referring again to FIG. 1, during shutdown of the treatment device 2, the first steam valve 36 and the condenser outlet valve 70 automatically close to prevent any reverse flow into the sealed areas of the system. Similarly, the first and second positive displacement pumps (8 and 10) shut down in a manner that prevents any fluid communication and therefore act as closed valves when not operational. This is beneficial in the event of an unplanned shutdown as no external fluid can enter and contaminate the sealed areas of the system. The temperature of the first and second heater elements (26 and 54) reduces any steam within the system and cools and condenses to create a vacuum inside the degas module 4 and the evaporator module 6. As discussed above, this ensures sterile conditions are maintained during a planned or unplanned shutdown of the device 2. As a further safeguard, the positive displacement mechanism (e.g. peristaltic or elliptical rotor) of the second pump 10 isolates the degas module 4 from the evaporator module 6.

After boiling and evaporating about 2 litres of water in the second boiling vessel 50, about 200 ml of water maintain. This water has a relatively high concentration of impurities. For example, the impurities from 2 litres of water remaining within a residual 200 ml will have impurities concentrated by a factor of about 10:1. When the treatment device is next used and boils another 2 litres of water for treatment, the 200 ml remain in the second boiling vessel 50 will have an impurity concentration of around 20:1.

To mitigate against these problems, the master control unit 40 incorporates a reverse flush functionality in which the first and second pumps 8 and 10, being positive displacement pumps such as peristaltic pumps, are run in reverse to draw the contaminated water out of the first and second boiling vessels (24 and 50) and into the supply tank 16 or waste tank 43. In this reverse operation, contaminated water from second boiling vessel 50 is restricted from flowing back into first boiling vessel 24 by valve 48 and is instead drawn though second gas outlet 39 to waste tank 43. Water in first boiling vessel 24 is drawn directly back through fill tube 44 to water supply 16. This avoids significant scalp deposits building up on the surface of the heaters (26 and 54) or corroding the components within the degas module 4 and evaporator module 6. Instead the reverse flush water with high concentration of impurities is simply flushed out of the hot water supply tank 16 and/or waste tank 43 by the user immediately before and/or after each treatment cycle.

Alternatively or additionally, contaminated water in second boiling vessel 50 may be removed by an additional pump or back through first boiling vessel 24 when outlet valve 48 is removed or bypassed. Referring now to FIG. 8, there is illustrated an alternate embodiment system 200, which includes a third pump 202. Pump 202 is controlled by controller 40 to draw liquid and contaminants from second boiling vessel 50 via outlet 204 at predetermined times. The liquid and contaminants are drawn along drain path 206 to waste or back to reservoir 43. The pump cycle and flow rate of pump 202 is controlled by master control unit 40 and is based at least in part on the temperature and conductivity of the liquid in second boiling vessel 50.

Master control unit 40 may be configured to control the flow and direction of pumps 8 and 10 and/or activate pump 202 in predefined cycles to pulse the flow of liquid in the system. By way of example, a purge cycle may involve running the purge pump 202 (or pumps 8 and 10 in reverse) for 2-3 seconds then running the system as normal (forward direction) again for 10-20 seconds and repeating this cycle as necessary. The purge process may be performed at predetermined intervals such as every 100 seconds.

In system 200, no non-return valves are needed between degas module 4 and evaporator module 6 as second pump 10 is not operated in reverse. Further, an outlet 208 is connected to a pressure release valve 210 to vent steam to atmosphere when a pressure in second boiling vessel 50 reaches a threshold pressure (e.g. 0.7 bar). This threshold pressure should be set to be at a maximum safe operating pressure of second boiling vessel 50.

In addition or alternatively, contaminants may be removed from evaporator module 6 through a blowdown procedure described below.

### Alternate Embodiment System

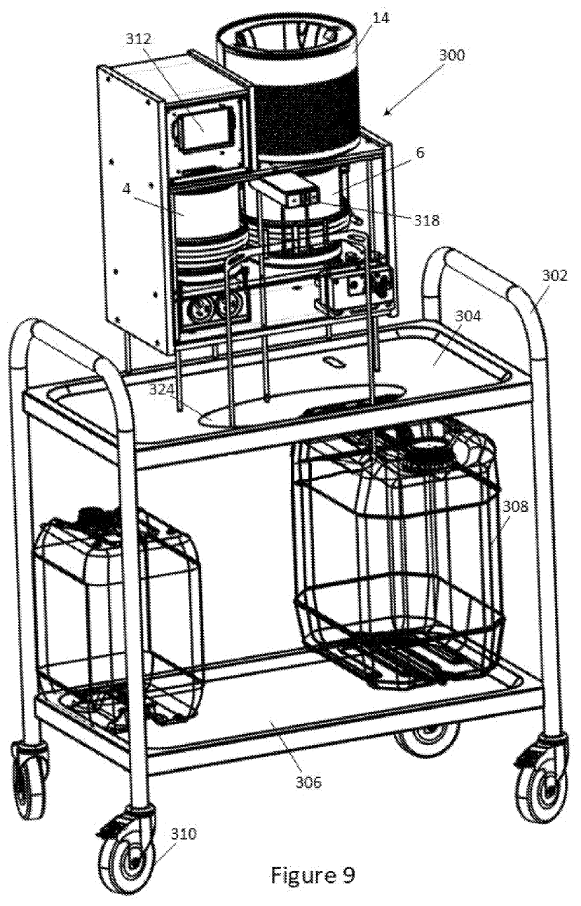

Referring now to FIGS. 9 to 12, there is illustrated a further embodiment fluid treatment device 300. Device 300 shares many similar components to systems 2 and 3 described above and common features are designated with the same reference numerals.

Referring initially to FIG. 9, device 300 is portable and capable of being situated on a portable trolley 302. Trolley 302 includes an upper support base 304 for supporting device 300 and a lower support base 306 for supporting fluid containers e.g. 308 or other items. Trolley 302 includes wheels e.g. 310 for allowing portability of device 300. However, in other embodiments, device 300 is able to situated on a bench top or other surface. Device 300 may source water locally from containers 308 or may have an inlet that allows the device to be plumbed to a mains water source.

Where device 300 is connected to mains water, first pump 8 may not be required to pump water into degas module 4 and may be replaced with a solenoid valve or similar.

Device 300 includes a display interface 312, which may be a touchscreen interface. Interface 312 is connected to master control unit 40, which is housed behind interface 312.

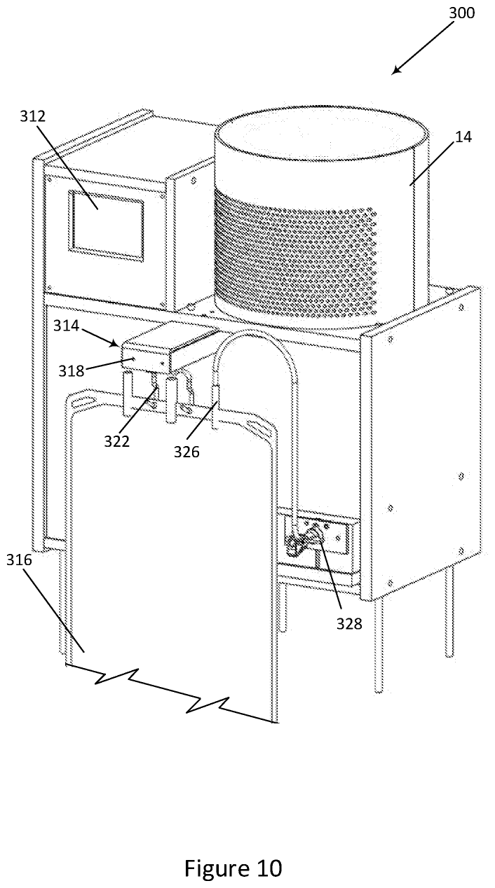

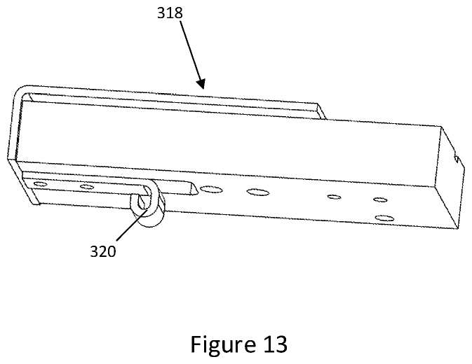

As best shown in FIGS. 10 to 13, device 300 includes a bag support and weighing system 314 for supporting a bag 316 to be filled such as a dialysate bag containing concentrated dialysate. Support and weighing system 314 includes a load cell weight sensor 318 mounted to device 300 and extending outwardly therefrom. As best shown in FIG. 13, an underside of weight sensor 318 includes a supporting hook 320 for supporting a bag support wire 322, as illustrated in FIGS. 10 to 12. Bag support wire 322 is generally formed into an inverted U shape with two ends bent outwardly and upwardly to allow a pair of supporting holes of bag 316 to be sleeved thereon and held in position.

When supported on support and weighing system 314, bag 316 hangs vertically downward as shown in FIGS. 9 to 12. As shown in FIG. 9, a hole 324 is formed in upper support base 304 to allow bag 316 to be fully extended. Weight sensor 318 senses the weight of bag 316 initially and as it is being filled with treated liquid from device 300, and sends a sensor signal to the master control unit 40.

During filling of bag 316, an inlet port 326 is connected to a sterile coupling device 328, which couples to outlet 73 of condenser 14. The operation of coupling device 328 is described below.

Bag support and weighing system 314 serves to perform a number of functions, including:
Supporting the bag 316 in position to be filled by device 300.
Zeroing the bag by detecting a stable reading over a few seconds.
Performing an initial check of the bag weight to ensure the bag is correct and contains the correct amount of concentrate (e.g. dry powder dialysate). An approved range of weights may be pre-programmed into the memory of master control unit 40.
Facilitating authorisation of the bag to be filled via the master control unit 40 upon sensing the correct weight bag.
Facilitating monitoring of the filling of the bag by sending signals from weight sensor 318 to master control unit 40. Through calibration, the sensed weight can be converted to a fill percentage of the bag.
Detecting the filling of the bag and facilitating shut-off of the flow of fluid from device 300.
Detecting anomalies in the weight of the bag or instantaneous rate of filing and facilitating shut-off of the filling.

In some embodiments, two or more weight sensors are used in parallel for redundancy and for greater accuracy.

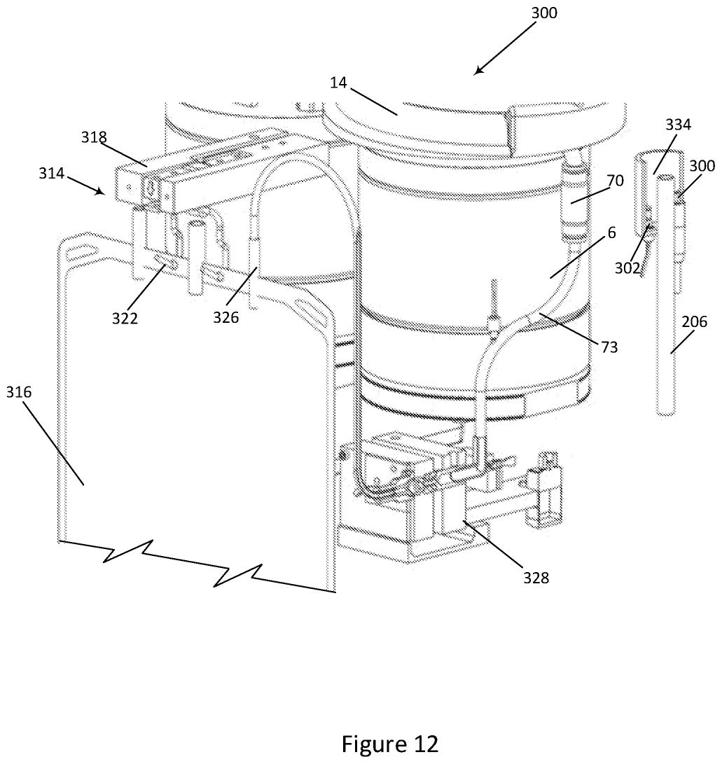

Referring to FIG. 12, drain path 206 from pump 202 includes a conductivity sensor 330 and a temperature sensor 332. Both sensors 330 and 332 are located within a reservoir 334 in which liquid and contaminants from second boiling vessel 50 can be situated during a bleed out process. Sensing conductivity at this point serves as a proxy for how dirty or contaminated the fluid is in the second boiling vessel 50. This sensing of temperature and conductivity can be fed back to master control unit 40 to control the flow rate and pump cycle of pump 202. By way of example, the speed of the pump cycle may be increased to perform higher agitation of the fluid in the second boiling vessel 50, tubing and/or connection points to clear out more contaminants.

### Self-Sterilising Coaxial Connector

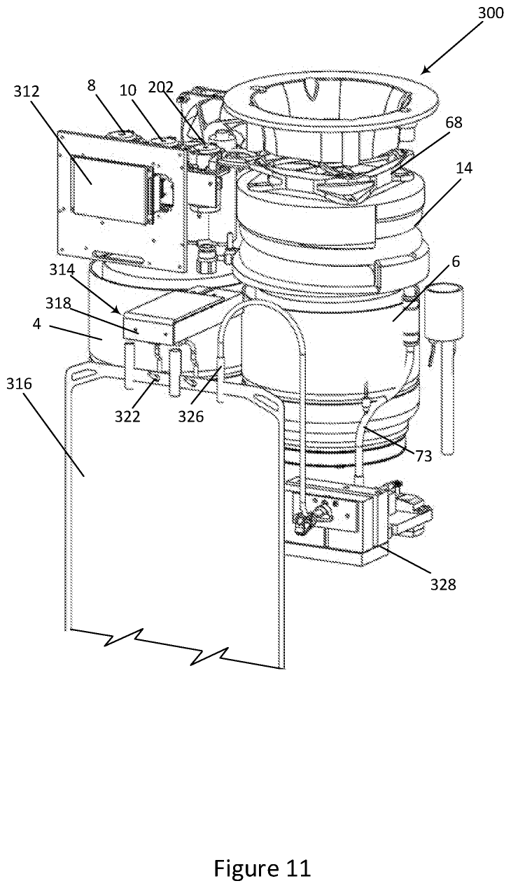

Referring now to FIGS. 14 to 17, there is illustrated a self-sterilising connection system 400 which uses coupling device 328. System 400 uses steam from condenser outlet tube 73 to perform surface decontamination of the exposed surfaces of the connector itself and of a connector of a patient device such as a bag/container of concentrated medicament, as illustrated in FIGS. 11 and 12.

To achieve this, coupling device 328 includes a first mounting portion 402 adapted to receive outlet tube 73 in fluid communication with a first coupling formation 404. In the illustrated embodiment, first mounting portion 402 takes the form of a substantially rectangular block that is fixedly mounted to a base 406 and having aperture 408 through which outlet tube 73 and first coupling formation 404 are disposed.

A second mounting portion 410 is adapted to releasably engage with a connector 412 of the patient device having a second coupling formation 414. Second mounting portion 410 is substantially similar in overall shape to that of first mounting portion 402 and the portions have respective opposing flat faces 416 and 418. As best shown in FIG. 14, second mounting portion 410 has an aperture 420 through which connector 412 is inserted.

In the illustrated embodiment, first coupling formation 404 is illustrated as a male type luer connector and second coupling formation 414 is a swabbable female type luer connector. However, in other embodiments, the male and female portions may be reversed and other types of fluid connectors may be used.

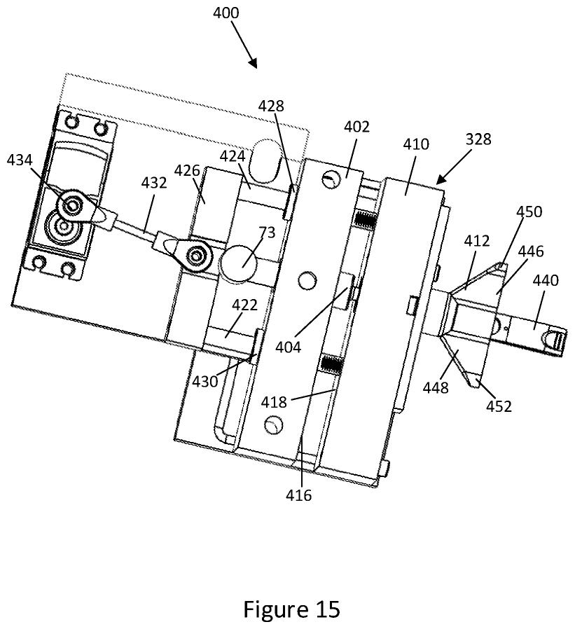

As shown in FIG. 15, two parallel linear shafts 422 and 424 extend from an actuating block 426 and project through bearings 428 and 430 of first mounting portion 402 and into second mounting portion 410.

System 400 includes an actuation mechanism in the form of actuator arm 432. Arm 432 is configured to linearly slideably move second mounting portion 410 between a first open position shown in FIG. 15 and a second engaged position shown in FIG. 14. In the first open position, the first and second coupling formations 404 and 414 are not touching but held within an at least partially enclosed space in close proximity. The partially enclosed space is defined by opposing faces 416 and 418 of mounting portions 402 and 410. In this position, coupling formations 404 and 414 are maintained within a distance of about 2 mm to 10 mm of each other.

In the second engaged position of FIG. 14, first and second coupling formations 404 and 414 are connected in sealing engagement to form an internal fluid channel. This allows treated fluid to flow from outlet tube 73 to connector 412 and on to bag 316 to be filled.

Linear sliding movement between the first and second positions is achieved by rotation of a rotating head 434, which pushes or pulls actuator arm 432 to move actuating block 426. This, in turn, moves second mounting formation 410 via the linear shafts 422 and 424. In some embodiments, rotating head 434 may be actuated to rotate manually via user operation. In other embodiments, rotating head 434 may be actuated automatically via an electrically controllable servo motor or other electromechanical actuation device.

In the open position of FIG. 15, steam or hot water may be expelled from outlet tube 73 to pre-clean the coupling formation 414 of connector 412. By switching off the condenser fan of device 300, steam can be generated from outlet tube 73, and the steam can be directed onto the exposed second coupling formation 414 so that it washes the exposed surfaces on both first and second coupling formations 404 and 414. As shown in FIG. 14, a drip tray 438 is disposed below mounting portions 402 and 410 to receive condensed liquid produced during the steam sterilisation process.

After this pre-cleaning or self sterilisation, the system 400 is moved into the engaged position of FIG. 14 to form a fluid connection between outlet tube 73 and connector 412. When the condenser fan is turned on, the steam will be replaced with purified water. This is synchronised with the movement from the open first position to the engaged second position to facilitate filling of the attached bag.

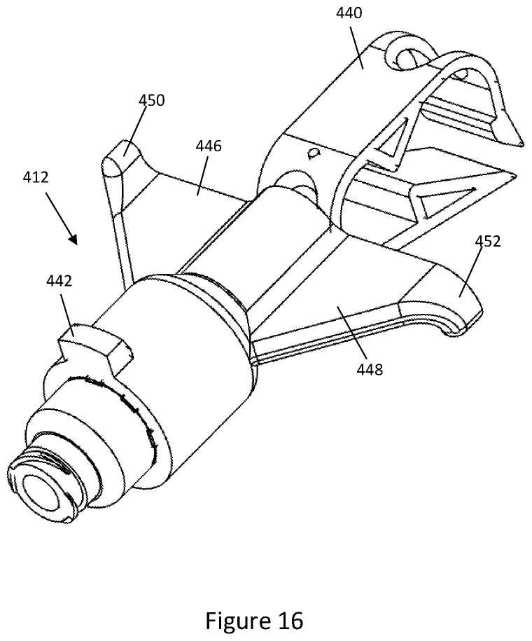

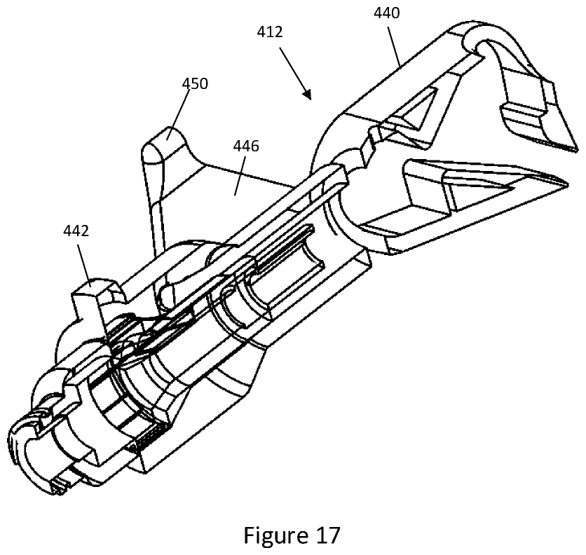

Referring now to FIGS. 16 and 17, connector 412 is attached to a tubing clamp 440 for selectively clamping or opening a tube connecting to the bag. Connector 412 include a bayonet type locking protrusion 442. When inserted into aperture 420 in the correct orientation, locking protrusion 442 aligns with and is received into a guide aperture 444 in second mounting portion 410, as best shown in FIG. 14. A corresponding guide slot is provided in second mounting portion 410 for allowing for rotatably sliding the locking protrusion into a locked position under rotation of the connector.

To facilitate locking rotation of connector 412, connector 412 includes wings 446 and 448 as shown in FIGS. 15 to 17, for allowing a user to grip and apply torque to connector 412. These wings include respective upward lips 450 and 452 that extend in opposing directions to guide a user on which direction to turn connector 412.

Although not illustrated, second coupling formation 414 includes a silicon “plug” often described as a duck-bill valve which is closed normally, and opens when the connection is made in the second engaged position.

Although system 400 is illustrated as operating in conjunction with a fluid distiller 300, it will be appreciated that system 400 may be used with other fluid treatment systems such as filter systems or reverse osmosis systems.

### Dialysis Preparation System

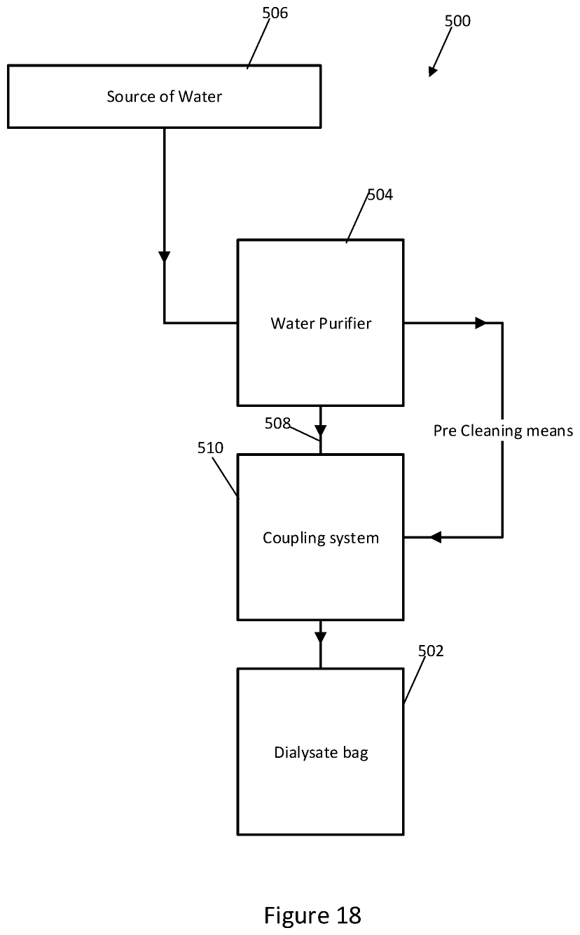

With reference to FIG. 18, there is illustrated a system 500 for filling a dialysate bag 502 containing concentrated dialysate from a water purifying device 504. The filled dialysate bag may be used, for example, to perform peritoneal dialysis treatment.

Device 504 may be a distiller such as the systems and devices described above. Alternatively, device 505 may include a water ultra-filter, a reverse osmosis system and/or a de-ionisation system. Device 504 is connected with a source 506 of water such as mains water or a water tank.

Device 504 includes a fluid outlet 508 for outputting purified water to a sterile coupling system 510. In some embodiments, sterile coupling system 510 may include coupling device 328 described above. Sterile coupling system 510 is, in turn, connected to a fill port of dialysate bag 502. Sterile coupling system 510 may perform automatic coupling between fluid outlet 508 and dialysate bag 502 so as to avoid or minimise risk of contamination by a user. Sterile coupling system 510 may also perform sterilisation of the coupling such as by using steam from the water purifying device 504 as described above.

System 500 may also include a controller which controls the rate at which water is passed from water purifying device 504 through coupling system 510 and into dialysate bag 502. The controller may also facilitate the automatic connection and disconnection of coupling system 510 and optionally perform sterilisation of the coupling.

System 500 allows for the safe and sterile filing of dialysate bags in the home or in remote locations from an untreated water source. The dialysate bags are pre-filled with concentrate such as dry powder and can be efficiently shipped to domestic or remote locations due to their smaller size and weight.

System 500 may also be used with other types of medicament containers other than dialysate bags that contain concentrated medicament.

### Device and System Operational Mode Summary

In the interests of clarity, example operating modes of the treatment device 2 and the peritoneal dialysis system are set out below in a tabulated form. The component nomenclature used here corresponds to that shown on the system diagram of FIG. 1 rather than the equivalent terminology of the components used throughout the detailed description. For example, DGM is degas module, EVM is evaporator module, P1 is first pump, P2 is second pump, F1 is fan, H1 is degas heater, H2 is evaporator heater, VT1 is degas vent valve temperature sensor, L1 is the first liquid level sensor, L2 is second liquid level sensor and so on.

Start-up mode

| | |
|---|---|
| Fill DGM | operate P1 until L1 is satisfied |
| Heat DGM | operate H1 at maximum power until T1 (in fluid) is at about 80° C. |
| | reduce H1 power proportionately as T1 increases from 80° C. to 105° C. |
| | this prevents “violent boiling” spitting water out vent |
| | after water reaches boiling point, Vent Temperature VT1 will increase |
| | above ambient - e.g. to an illustrative target half way from ambient to boil |
| | (100-20) = 60° C. |
| Establish GAS | when VT1 achieves 60° C., control method transitions from Start-up mode |
| VENTING OUTPUT | to Operate mode |
| P2 stays off (no water | |
| moved to Evaporator) | |
| Weight Platform | check platform empty and “zero” weight calibration |
| Load platform | user places empty bag on platform, threads “fill” tube through neutral |
| | axis of loadcell. Check bag weight conforms to intended prescription. |
| | E.g. 2.5% dextrose should be 77-78 grams |

Operate Mode

| | |
|---|---|
| H1 | power level modulated to maintain VT1 at target |
| P1 | operational speed controlled to maintain L1 in evaporator using smooth |
| | PID control (the meaning and relevance of PID control is described |
| | below) |
| L1 | “error” signal generated from level sensor or similar level sensing method |
| | or algorithm provides smooth control of P1 |
| P2 | operation is enabled once VT1 has been at or above target level for |
| | more than 30 sec |
| | P2 throughput is controlled by L2, typically substantially filling the |
| | evaporator at maximum speed until the heater surface is covered, then |
| | reducing to modulation under PID control |
| P1 anticipation | in additional to P1 control by PID as described, when P2 starts, P1 |
| | control algorithm steps up output to match the draw-off by P2 |
| VT1 anticipation | When P1 is operational, in addition to PID, H1 output is boosted to |
| | compensate for cooler fluid added by P1 |
| Operate Mode - | Bag coupling not detected |
| standby | |
| F1 | condenser fan speed adjusted to maintain low temperature just above |
| | ambient (e.g. 40° C.) |
| H1 heater power | H1 power adjusted to achieve F1 fan speed of minimum (5% airflow rate) |
| minimum | |
| Bag Weight Sensor/fill | confirm “no bag” on platform and “zero” calibration of sensor (warn user |
| sensor | if bag present) |
| Operate Mode - sterilise | Bag coupling detected |
| F1 | fan speed OFF until sterile coupling temperature of 80° C. achieved |
| | this sterilise phase continues for 30 sec after 80° C. is achieved |
| Bag Weight Sensor/fill | confirm weight of bag is correct for prescription (e.g. it should be 1.5% or |
| sensor | 2.5% or 3.35% or 4.25%) |

Operate Mode - fill

| | |
|---|---|
| F1 | Condenser fan speed adjusted to MAXIMUM speed |
| H2 | heater power adjusted to achieve condenser fluid output temperature of |
| | 90-95 C. |
| Bag Weight Sensor/fill | monitor bag weight until 2.0 L confirmed |
| sensor | |

End Sequence

| | |
|---|---|
| H1 and H2 power off | when platform weight has increased by 2000 gr + allowance for tube |
| Non Return valve at | volume |
| vent out closes | |
| Non Return valve at | |
| condenser out closes | |
| User instructed to | |
| remove bag and cap | |
| outlet | |
| P1 and P2 operated in | this is equivalent to the term “blowdown” used earlier and explained in |
| reverse | detail below. Dirty water in both boilers returned to cold water tank. |
| Finish | A platform is provided with a load-cell (measures weight) |
| | On start-up check it is empty with optical sensor and re-adjust calibration |
| | to set zero. |
| | User places empty bag on platform. System weighs the bag and confirm |
| | it is correct (e.g. of prescription is for 2.5% dextrose, bag should weight |
| | about 78 gr |

### PID Control

PID is common shorthand for a control method “Proportional Integral Differential” (as distinct from On/Off control).

Consider the pump P1 filling the DGM. If using On/Off control, the pump operates at full speed for a while, then is Off for a while. When “On” the incoming slug of cold water will quench the boiling process. As the system is then momentarily without steam being ejected, the system may not degas. The slug of cold water also introduces the risk of it migrating unmixed to the outlet.

A similar situation exists with the heater. If using On/Off control, when the heater is off, no steam is being produced. It is preferable to match the first pump speed (flowrate) and heater power output to exactly the operating requirement.

One problem is, if the pump and heater are designed to be exactly right, there is no provision for any small system variability. It would be difficult therefore to predict a single fixed value for either. Further, on startup, it is desirable to have a first pump with significantly higher flowrate than design.

At “design” flowrate—e.g. to fill a 2 L bag in 1 hour, the flowrate is 2 L/hr. The DGM and second boiling vessel 50 of the evaporator module 6 have volumes of about 250 ml each, therefore, it would take 15 minutes just to “pre-fill” the system.

The first pump of the present invention has a capacity of 5× design (10 L/hr) and therefore can “startup” in 3 minutes.

The degas heater power-in stable operation would be about 200 W, but, use of a heater with “full power” or 1,200 W is possible in the present invention, which again allows very fast start-up.

Because the first pump 8 and first heater 26 are overrated by 500% or more, on/off control would result in major swings in performance.

To implement, PID control relies on two pre-conditions.
1. The measured parameter should be a variable (for example, the water level sensor L1 cannot be simply a level switch with an On/Off binary signal). This is why the capacitive sensor is preferred where the water level acts as a variable dielectric, giving an output signal which is proportional to water level. This keeps the system self-calibrating both at “zero” and “range”.
2. The controlled parameter (e.g. pump) can provide a variable output (or quasi variable). Microprocessors will use PWM (pulse width modulation) to control the pump speed. The pumps are fed with a square wave voltage rapidly switching on/off. The frequency is higher than the mechanical response time of the motor. The ratio of “on” time to cycle time determines the resultant motor speed.

### Blowdown

Blowdown is a term used in dealing with pressurised steam boilers. A boiler is characterised by the presence of a layer of liquid water (incompressible) and a layer of steam above it (compressible). While still under pressure, a fluid connection at the lowest point in the boiler is opened to the outside environment of ambient pressure. The pressure of steam rapidly expels the water, and due to the energy available, will also often transport particulate matter (scale etc). The steam can expand as the water is expelled, maintaining a positive outflow. It can be a partial blow-down, where only a portion of the water is removed, or a full blowdown where all water is expelled. This allows the highly concentrated process water to be removed, and replaced with clean water.

In some embodiments of the present invention, rather than relying on pressure, the system uses the pumps to expel the water as described above in relation to flushing the system.

However, the system may be configured to implement a blowdown process to remove contaminants from second boiling vessel 50. Referring to FIG. 1, when a blowdown procedure is run in second boiling vessel 50, second heater 54 is activated to significantly and rapidly increase the heat energy delivered, increasing the amount of steam evaporated and thereby the pressure within boiling vessel 50. When a sufficient high pressure is reached, a pressure operated flush valve 67 is activated to draw the water and steam out of a pressure release outlet 69. Outlet 69 is located at a lowest point in second boiling vessel so as to promote the egress of fluid and gases through outlet 69. The contaminated water drawn out of outlet 69 via flush valve 67 is vented to atmosphere if the pressure in second boiling vessel 50 is above a threshold pressure (e.g. 0.7 bar). To complete the blowdown process, second heater 54 is powered down to a lower temperature or deactivated.

### System Optimisation and Control

The control of heaters 26 and 54 occurs via respective heater controllers 32 and 66, which are in turn controlled by master control unit 40. Heater control in devices 2 and 3 must deal with two problems: power availability and power distribution. A typical domestic power outlet is limited to loads in the range of about 1,500 W (countries with 100 V to 120 V supplies) up to about 2,200 W-3,000 W in other regions.

Upon system startup, it may be desirable to minimise startup time. This can be achieved by arranging for the degas module 4 to use all the available power. Once the degas module 4 has heated and is boiling, the module of size described above needs only a theoretical 15 W. However, allowing for heat losses, still only in the region of 65 W to maintain an effective outflow of gas, VOCs and steam. As water is drawn off to the evaporator module 6, the need to heat the incoming fluid can increase the power requirement to typically 250-300 W. The remaining power can then be deployed to the evaporator module 6.

The master control unit 40 can be programmed with control algorithms to define a power “cycle” say 3 sec. These algorithms receive feedback from the various temperature and level sensors to determine a required power for each heater. For the evaporator module 6, this is preferably done by a PID method sensitive to the temperature of a thermally conductive surface in fluid communication with the outflow of steam and gas on one side of the surface and ambient air on the other. As the controller increased the heater power, producing more steam, this temperature will tend towards 100° C.

This feedback, in the case of an illustrative setpoint of 85° C., would result in the controller reducing power to achieve setpoint. If the steam supply is very low (e.g. rapid flow of cold incoming water to the degas module 4) the sensed temperature will tend to drop towards ambient, and the sensor/control system will immediately increase power. The inventor has found that the responsiveness of the system can be enhanced by adding an “anticipation” method. As the pump speed is varied (increased or decreased), a small reset of the control setpoint can anticipate the effect of the changed pump speed. In software, this method can also be deployed in the form of a shift in the calculated target power output of the heater, if flow is increased by, say 10%, heater power would be proportionally increased (about 20 W for each 10% change in pump flowrate).

Cost of both manufacture and operation is a significant consideration. In fixing the system parameters, maximum performance is sought for a given fixed configuration. For a given boiler size, there is little cost difference as the heater power output is chosen to be higher or lower than a nominal size. In manufacturing terms, a higher power heater is often lower cost. The heat generating coil is wound from a length on NiCrFeAl resistance wire. When lower powers are required the wire needs to be thinner and longer to achieve a higher resistance. This increases manufacturing cost and the thinner wire is less reliable, and susceptible to penetration damage from the compressed MgO powder surrounding it. If two 2.2 KW heaters are chosen for the first and second heaters 26 and 54, optimum cost can be achieved, but this would exceed the power limits of a single domestic or similar power outlet. Thus, cost of design must be balanced with operational requirements like power consumption for domestic and medical use.

The cost of the condenser 14 impacts the system. As the condenser is air cooled, it requires a large surface area to be effective. The heat transfer coefficient of such a surface can typically be 10-12 W/m²/° C. in still air. When air is drawn across a bank of circular tubes with circular aluminium cooling fins, the air will take the path of least resistance, with most of the air flowing through the tangential gap where the periphery of the aluminium fins meet, and little or no air penetrating into the gap between the fins to the surface of the inner tube. In fact, in the direction of flow, there is a stagnation point at the tube surface on the inflow and outflow direction.

The inventor has sought to optimise the heat transfer by directing the air to achieve a consistent air velocity across the fins by blocking the flow that might otherwise follow a path tangential to the fins.

The condenser 14 must have sufficient capacity to provide the required fluid flow under worst case conditions (such as when ambient temperature is 35° C.).

The inventor however also considers the performance of the condenser considering the thermodynamic performance. The coefficient of heat transfer of condensing steam on a stainless steel surface is in the region of 50,000 to 100,000 W/m² (depending of gas velocity, and condensate film thickness). As water condenses within the condenser tube, the heat transfer to the wetted surface drops considerably, to a value in the range 800-1,600 W/m²/° C. This implies that whereas condensation can be achieved reasonably efficiently, using the condenser as a means of cooling the water is less efficient.

One problem is that if the condenser 14 is selected to just cope with the system load at high ambient temperature, there is a danger that such temperature might be exceeded under exceptional circumstances. Additionally, there are risks, such as a user somehow blocking the airflow across the condenser, and air filter, or condenser surface having efficiency reduced by dust or other contamination. The selection of condenser could be sized to cope with these worst case parameters, but, for the average user in optimal circumstances, the system would appear to be cooling the outgoing water to close-to-ambient temperature, and with a flowrate of only half what it might otherwise be.

The inventor has devised a means to overcome these problems, maintaining maximum throughput, and also ensuring that the outgoing fluid is sufficiently treated by adding a feedback loop. A temperature sensor 72 is positioned in the outlet fluid flow from the condenser.

Temperature sensor 72 provides two functions. First, on start-up, the condenser fan is switched off, the sensor 72 detects the initial flow of hot fluid from the condenser, and initiates a timer. It has been found that by maintaining high power in the second heater 54, an outflow of condenser 14, which consists of a single contiguous flow path with minimal re-entrant features and a steam velocity greater than 5 m/s, can reduce the bio burden in the outflow to virtually zero. In this phase, the second heater 54 power is set at a predetermined (maximal) power of around 1,200 to 2,200 W minus the power of first heater 26, resulting in a power typically in the range 800-1,800 W.

Sensor 72 can then serve a second critical function, as the system changes over. The fan is started up to maximise condenser output. As the condenser will typically have a nominal capacity of about 750 W, a setpoint will be established for the outgoing condensate slightly below condensation temperature—typically in the range of 85-95° C. The measured temperature value is then used to control the power input to the second heater 54 to maintain this target. If the temperature falls to the lower end of the range, the power will be proportionally increased, or preferably controlled by a PID method to achieve optimal control. This method has the dual benefit of automatically achieving and maintaining sterile conditions while also continuously matching performance to changing ambient conditions.

In spite of such precautions, there can be occasions when biofilm growth can result is challenging circumstances where the bio burden remains detectable. The inventor has realised that the specific heat of steam is quite low (˜2 KJ/kg/° C. as compared with 2,400 KJ/kg latent heat). The flowline system design of the present invention facilitates the addition of an optional small heater in the outgoing steam flow (prior to the condenser). A power of only 40 W is sufficient to superheat the dry steam to temperatures in excess of 180° C., or indeed the same method can heat to 250° C. if required, and thus purge all surfaces in the condenser and downstream with a thermal sterilizing means.

### Conclusions

The embodiments of the invention described above provide a hand-portable device that performs treatment of liquids such as water, and which is suitable for consumer use in the home or for medical use. The device operates off low power and can be powered by a standard mains power supply. Various embodiments of the device can be designed to weigh less than 25 kg, 15 kg and even less than 7 kg. However, larger scale devices may be designed also, such as versions suitable for small clinics.

Alternate treatment systems enter an inactive state when flow demand is reduced, which can produce a warm and moist environment for unwanted bacterial growth within the system. The treatment system described herein is designed as a continuous flow system wherein all components are capable of being operated with a varying continuous flow such that treated water can be dispensed while input water continues to be processed. This design avoids or substantially reduces possible bacterial growth within the system as the system is able to be maintained in a low flow rate (e.g. 3% of nominal system output).

The simultaneous control of pumps 8 and 10, and heaters 26 and 54 by master control unit 40 allow for continuous flowrate control to be performed to adjust the output flowrate of devices 2 and 3 from close to zero (e.g. 3% of system design output) through to a significantly large output such as 150% of nominal output.

As the incoming water temperature may also vary (commonly between about 5° C. to 30° C.), the energy needed to pre-heat this water to 100° C. in the degas module 4 can increase by 30% (representing about 7% of overall system load). The present invention is easily able to manage this variation in input temperature through the control of pump flow rates and heater control using master control unit 40.

The device is designed as an ‘in-line’ device. A continuous flow process which advantageously allows additional stages or process steps to be introduced to the existing two-stage treatment. By way of example, filtering such as particulate filtering and carbon filters can be introduced before or after the degas process to further filter the water being treated.

### Definitions

The invention has been described herein by way of example only. Skilled workers in this field will readily recognise mere variations and modifications which do not depart from the spirit and scope of the broad inventive concept.

Reference throughout this specification to “one embodiment”, “some embodiments” or “an embodiment” means that a particular feature, structure or characteristic described in connection with the embodiment is included in at least one embodiment of the present disclosure. Thus, appearances of the phrases “in one embodiment”, “in some embodiments” or “in an embodiment” in various places throughout this specification are not necessarily all referring to the same embodiment. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

It should be appreciated that in the above description of exemplary embodiments of the disclosure, various features of the disclosure are sometimes grouped together in a single embodiment, Fig., or description thereof for the purpose of streamlining the disclosure and aiding in the understanding of one or more of the various inventive aspects. This method of disclosure, however, is not to be interpreted as reflecting an intention that the claims require more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this disclosure.

Furthermore, while some embodiments described herein include some but not other features included in other embodiments, combinations of features of different embodiments are meant to be within the scope of the disclosure, and form different embodiments, as would be understood by those skilled in the art. For example, in the following claims, any of the claimed embodiments can be used in any combination.

In the description provided herein, numerous specific details are set forth. However, it is understood that embodiments of the disclosure may be practiced without these specific details. In other instances, well-known methods, structures and techniques have not been shown in detail in order not to obscure an understanding of this description.

1. A multistage treatment system for a liquid, the system comprising:
a first stage comprising a first boiling vessel and a first heater configured to boil the liquid in the first boiling vessel at a first pressure, the first pressure being greater than ambient pressure, such that the boiling liquid generates vapour including volatile contaminants, a first vapour outlet configured to vent the vapour from the first boiling vessel, and a liquid outlet for fluid flow from the first boiling vessel to a subsequent stage;
a second stage comprising a second boiling vessel having a liquid inlet to receive liquid from the first liquid outlet and a second heater to boil liquid in the second boiling vessel to generate vapour, the vapour being output via a second vapour outlet in the second boiling vessel;
a condenser to receive the vapour from the second vapour outlet and condense the vapour to a liquid that is treated, and
a primary outlet configured to dispense treated liquid.

2. The system according to claim 1 further comprising a heat exchanger having a first end connected to the first vapour outlet and a second end connected to ambient air, wherein the heat exchanger includes a first temperature sensor disposed at a location in the heat exchanger where the temperature is approximately half way between an internal temperature at the first boiling vessel and an output temperature.

3. (canceled)

4. (canceled)

5. (canceled)

6. (canceled)

7. The system according to claim 1 wherein the first stage includes a first fluid supply device for drawing the liquid from a source and supplying liquid to the first boiling vessel.

8. (canceled)

9. (canceled)

10. (canceled)

11. (canceled)

12. (canceled)

13. (canceled)

14. The system according to claim 1 wherein the first stage has a liquid temperature sensor and a vapour temperature sensor, such that during use, output from the liquid temperature sensor and the vapour temperature sensor is used for feedback control of the first heater.

15. The system according to claim 2 wherein control of the first heater is based at least in part on a temperature sensed by the first temperature sensor.

16. The system according to claim 7 wherein the first stage includes a degas module and the second stage includes an evaporator module, the evaporator module being in fluid communication with the degas module via a second fluid supply device such that the second fluid supply device draws the liquid from the first boiling vessel to the second boiling vessel.

17. (canceled)

18. The system according to claim 16 further comprising a third fluid supply device in fluid communication with the second boiling vessel and configured to move liquid and contaminants from the second boiling vessel at specific intervals.

19. (canceled)

20. The system according to claim 16 wherein the evaporator module includes one or more evaporator liquid sensors and wherein one of the evaporator liquid sensors is positioned to detect when the second heater is immersed in the liquid.

21. The system according to claim 16 wherein the second fluid supply device is disabled until the first temperature sensor in the degas module reaches a predefined threshold temperature and a predefined amount of time has passed at or above this threshold temperature.

22. (canceled)

23. The system according to claim 1 wherein at least one of:
the system is configured to operate on a total input power of less than or equal to 2,400 W; or
the system is powered by a mains power supply.

24. (canceled)

25. The system according to claim 16 wherein the first and second fluid supply devices are:
positive displacement pumps configured to prevent liquid flow when not operating; or
peristaltic pumps configured to prevent liquid flow when not operating.

26. (canceled)

27. The system according to claim 16 wherein the system further comprises an output heat exchanger for receiving outlet water flow from the condenser to heat inlet water flow to the degas module.

28. The system according to claim 1 wherein the primary outlet is configured for sealed fluid connection to a container containing concentrated solutes or dry powder concentrate to form a treatment solution with the liquid treated by the system.

29. (canceled)

30. (canceled)

31. (canceled)

32. The system according to claim 1 further comprising an output temperature sensor positioned in an outlet fluid flow of the condenser, the output temperature sensor configured to sense a temperature of fluid from the condenser, wherein the condenser includes a fan and the condenser fan is responsive to a signal from the output temperature sensor to increase or decrease the fan speed.

33. (canceled)

34. The system according to claim 1 wherein the system is configured to provide a continuous flow process in which treated liquid is dispensed concurrently while liquid is being processed in the first and second stages.

35. The system according to claim 1 including a mechanically actuatable sterile coupling device for moving the primary outlet into sterile coupling engagement with an inlet of a container of dried medicament or dry powder concentrate for producing dialysate.

36. The system according to claim 35 wherein the sterile coupling device is electromechanically actuatable into sterile coupling engagement with an inlet of the container of dried medicament or dry powder concentrate in response to a control signal.

37. The system according to claim 28 further comprising a weight sensor for sensing the weight of the container during a filling process.

38. A multistage treatment device for treating liquid, the treatment device comprising:
a first boiling vessel comprising a first heater for boiling the liquid in the first boiling vessel to generate vapour including volatile contaminants;
a first vapour outlet configured to vent the vapour from the first boiling vessel;
a liquid outlet for fluid flow of the liquid from the first boiling vessel to a second boiling vessel after a predetermined period of vapour flow through the first vapour outlet;
a second boiling vessel to receive the liquid from the first boiling vessel and comprising a second heater for boiling the liquid in the second boiling vessel to generate vapour; and
a condenser for condensing the vapour from the second boiling vessel to liquid that is treated, the condenser having a condenser outlet configured to dispense the treated liquid.

39. The system according to claim 7,
wherein the first boiling vessel includes a first liquid sensor for sensing when the first heater is immersed in the liquid, and wherein the first fluid supply device is selectively deactivated when the first liquid sensor senses the first heater is not immersed in the liquid,
wherein the first boiling vessel includes a second liquid sensor disposed at a higher position than the first liquid sensor in the first boiling vessel, and wherein output from the second liquid sensor is used to control the first fluid supply device,
wherein the system includes a controller configured to monitor a sensor signal from the second liquid sensor over a period of time to detect splashing of the fluid at a level of the second liquid sensor, and
wherein the controller is configured to detect a ratio of “on” time to total time of the sensor signal, wherein “on” time indicates that fluid is detected at the level of the second liquid sensor.

40-64. (canceled)
